(19) United States
(12) Patent Application Publication
(10) Pub. No.: US 2026/0355168 A1
(43) Pub. Date: Nov. 12, 2026

(54) COMPOSITIONS AND METHODS FOR TREATING AND DETECTING CANCER

(71) Applicant: CASE WESTERN RESERVE UNIVERSITY, Cleveland, OH (US)

(72) Inventors: Susann Brady-Kalnay, Wellington, OH (US); Kathleen Molyneaux, Cleveland, OH (US); Christian Laggner, Cleveland, OH (US)

(21) Appl. No.: 19/107,860

(22) PCT Filed: Aug. 29, 2023
(86) PCT No.: PCT/US2023/031406

§ 371 Date: Feb. 28, 2025

Related U.S. Application Data
(60) Provisional application No. 63/401,908, filed on Aug. 29, 2022.

(51) Int. Cl. A61K 31/4439; A61P 35/00

(52) U.S. Cl. CPC A61K 31/4439; A61P 35/00

(57) ABSTRACT

A pharmaceutical composition comprising a compound a binding pocket adjacent the wedge domain of an intracellular portion or fragment of the receptor protein tyrosine phosphatase (RPTP) IIb cell adhesion molecules (e.g., PTPμ) and that is capable of inhibiting RPTP mediated adhesion of cells and/or cancer cell growth and/or sphere formation.

### RELATED APPLICATION

This application claims priority from U.S. Provisional Application No. 63/401,908, filed Aug. 29, 2022, the subject matter of which is incorporated herein by reference in its entirety.

### BACKGROUND

Phosphorylation of specific amino acids (tyrosine, serine, threonine) in proteins is a well-known signal transduction mechanism for controlling protein function. This process, regulated by kinases and phosphatases, controls a wide-range of cell behaviors, including division and migration, that are important for development and normal physiology. However, disruption of kinase/phosphatase signaling cascades is a common feature in many disorders, including cancer. This has driven the development of therapeutic agents specifically designed to inhibit the catalytic activity of kinases, but attempts to target phosphatases, particularly tyrosine phosphatases, have lagged. The active sites of tyrosine phosphatases are both highly conserved and highly charged, meaning agents capable of targeting these sites in biochemical assays are often promiscuous and not suitable for in vivo use because they are unable to cross cell membranes. Thus, drug development in the phosphatase field has begun to focus on regulatory sites outside of the catalytic domain.

In this sense, the protein tyrosine phosphatase mu (PTPμ), a member of the IIb receptor protein tyrosine phosphatase (RPTP) family, is an attractive drug target. It has established structural motifs, defined by crystallography and deletion analysis, outside of its catalytic domain that could be exploited as targets, and it plays an important role in the development of several cancers. Structurally, PTPμ is member of a larger superfamily of RPTPs comprised of 21 genes in humans subdivided into types (Type 1/VI, IIa, IIb, III, IV, V, VII and VIII) based on the sequences of their extracellular and intracellular domains. Members of the IIb subtype, including PTPμ, have structurally conserved extracellular domains (with only a moderate 49-63% a.a. similarity) that contain an N-terminal meprin-A5-RPTPμ (MAM) domain followed by an Ig domain and four fibronectin type III repeats. The intracellular portion of PTPμ is comprised of two highly con-served phosphatase-like domains and a more divergent juxtamembrane region (resembling the cytoplasmic region of cadherins) predicted to have regulatory functions.

The extracellular domain of PTPμ mediates homophilic adhesion, with the MAM and Ig domains of one molecule interacting in trans with the first fibronectin repeat of another molecule. The Ig domain mediates homophilic binding directly in vitro. The MAM domain has been shown to mediate lateral (cis) interactions between PTPμ molecules within the same cell making an oligomeric functional adhesive complex. Engagement of adhesion via PTPμ is believed to be transmitted into changes in cell signaling via the catalytic activity of its membrane proximal phosphatase domain. Its second phosphatase domain is thought to be catalytically inactive but may have regulatory or, as shown for RPTPT, alternative enzymatic (denitrase) functions.

An additional regulatory structure, termed the wedge domain, is present within the juxta membrane region of a subset of RPTPs (LAR, PTPμ, PTPα, PTPδ, PTPσ and CD45), making it a more appealing target for specificity. The sequence of this region is more divergent than that of the tandem phosphatase domains and it has predicted regulatory functions. Mutations in the wedge domain of CD45 prevented dimerization-induced inhibition of CD45 activity, and the crystal structure of the membrane proximal and D1 phosphatase domains of PTPα provides a structural rationale for this. The wedge domain has also been shown to participate in interactions between the D1 and D2 phosphatase domains, interactions that have been shown to be inhibitory for some RPTPμ. Finally, the wedge domains may control the interaction of RPTPs with other binding partners leading to changes in downstream signaling. For instance, a LAR-wedge domain peptide was able to block the interaction of LAR with TrkA leading to activation of tyrosine kinase dependent signaling in PC12 cells. Likewise, a wedge peptide (Intracellular Sigma Peptide) directed against PTPσ was shown to affect signaling via Erks/CREB and RhoA/CRMP2 and is a promising agent for promoting neural regeneration after injury. Thus, it is possible that the wedge domain of PTPμ could control interactions with its binding partners/substrates, which includes cadherins, p120 catenin, PKCδ, PLCγ, IQGAP, and RACK1, with therapeutic potential.

Targeting the wedge domain of PTPμ is an attractive strategy for treating malignancies. PTPμ expression is reduced in several forms of cancer (prostate, ovarian, endometrial, melanoma, and glioblastoma). This suggests that PTPμ acts as a tumor suppressor, possibly by regulating adhesive interactions necessary for contact-dependent suppression of cell migration and/or growth. In some cancers, however, the loss of PTPμ is proteolytic, and both extracellular and intracellular fragments of PTPμ are retained in tumors. These fragments have been exploited to serve as prognostic biomarkers and imaging agents, but they are not just inert proteolytic byproducts. An shRNA-mediated reduction of PTPμ in a glioma cell line (LN229) (that expresses mostly PTPμ fragments) was shown to reduce cell migration and growth factor independent growth, suggesting the fragments have oncogenic activity. A small peptide directed against the wedge domain of PTPμ was also able to block migration and growth-factor independent survival of LN229 cells, indicating aberrant signaling via an intracellular fragment, which can accumulate in the nucleus, may drive these processes by interacting with inappropriate substrates. Importantly, the PTPμ wedge peptide did not interact with the LAR wedge region, suggesting this domain could be a highly specific drug target.

### SUMMARY

Embodiments described herein relate to compounds that target receptor protein tyrosine phosphatase (RPTP) cell adhesion molecules (e.g., PTPμ), and particularly a binding pocket bordered by the wedge domain of an intracellular portion or fragment of an RPTP, and that are capable of inhibiting RPTP mediated adhesion of cells and/or cancer cell growth and/or sphere formation and/or phosphatase activity as well as to their use in methods of treating cancer in a subject in need thereof and methods of detecting, monitoring, and/or imaging cancer cells and/or cancer cell metastasis, migration, dispersal, and/or invasion in a subject.

In some embodiments, the compound can have the structure of formula (I):

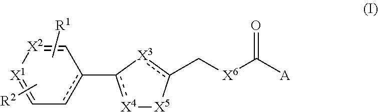

or a pharmaceutically acceptable salt, tautomer, or solvate thereof;
wherein,
a dashed line (e.g., ---- or --) is an optional bond;
A is cycloalkyl, aryl, heteroaryl, or heterocyclyl, each of which is optionally substituted with one or more R⁶;
X¹, X², and X³ are each independently C(H)ₘ, N(H)ₙ;
X⁴ and X⁵ are each independently N(H)ₙ or O;
X⁶ is CH₂ or N(R⁷);
R¹ and R² are each independently absent, ═O, halogen, hydroxyl, alkyl, haloalkyl, or alkoxy;
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocycle;
R⁷ and R⁸ are each independently H, halogen, hydroxyl, alkyl, alkoxy, or haloalkyl;
m is 0, 1, or 2;
n is 0 or 1; and
t is 0, 1, or 2.

In some embodiments, A is:

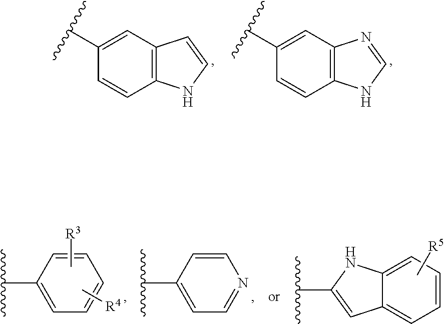

wherein,
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocycle; and
R⁵ is absent, halogen, hydroxyl, alkyl, haloalkyl, or alkoxy.

In other embodiments, the compound can have the formula selected from:

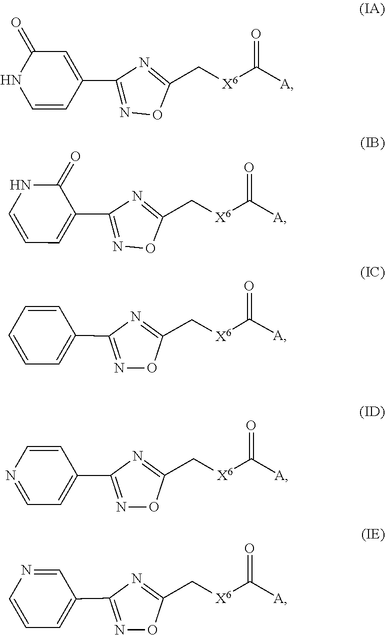

or a pharmaceutically acceptable salt, tautomer, or solvate thereof;
wherein,
A is cycloalkyl, aryl, heteroaryl, or heterocyclyl, each of which is optionally substituted with one or more R⁶.
X⁶ is CH₂ or N(R⁷);
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocycle;
R⁷ and R⁸ are each independently H, halogen, hydroxyl, alkyl, alkoxy, or haloalkyl; and
t is 0, 1, or 2.

In other embodiments, A is:

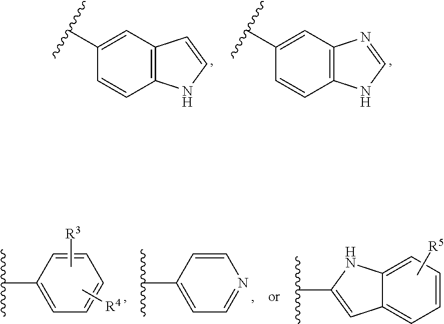

wherein,
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocycle; and
R⁵ is absent, halogen, hydroxyl, alkyl, haloalkyl, or alkoxy.

In other embodiments, the compound can have a structure of:

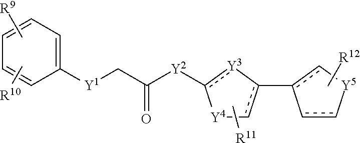

or a pharmaceutically acceptable salt, tautomer, or solvate thereof;
wherein,
a dashed line (e.g., ---- or --) is an optional bond;
Y¹, Y², Y³, Y⁴, and Y⁵ are each independently C(H)ₘ, N(H)ₙ, O, or S;
R⁹, R¹⁰, R¹¹, and R¹² are each independently absent, halogen, alkyl, hydroxyl, haloalkyl, alkoxy, —COOH, —C(O)—N(R¹³)₂, -alkylene-C(O)—N(R¹³)₂, -alkylene-OH, —C(O)O-alkyl, or -alkylene-COOH;
each R¹³ is independently H, alkyl, -alkylene-OH, —C(O)-alkyl, —C(O)O-alkyl, or -alkylene-COOH;
m is 0, 1, or 2; and
n is 0 or 1.

In other embodiments, the compound can have the formula:

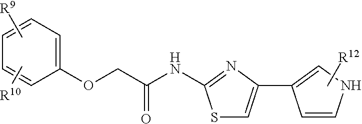

or a pharmaceutically acceptable salt, tautomer, or solvate thereof;
wherein,
R⁹, R¹⁰, and R¹² are each independently absent, halogen, alkyl, hydroxyl, haloalkyl, alkoxy, —COOH, —C(O)—N(R¹³)₂, -alkylene-C(O)—N(R¹³)₂, -alkylene-OH, —C(O)O-alkyl, or -alkylene-COOH; and
each R¹³ is independently H, alkyl, -alkylene-OH, —C(O)-alkyl, —C(O)O-alkyl, or -alkylene-COOH.

In some embodiments, the compound can be formulated in a pharmaceutical composition with a pharmaceutically acceptable carrier or excipient.

In some embodiments, the compound specifically binds to and/or complexes with an intracellular fragment or portion of an RPTP cell adhesion molecule, such as PTPμ, that is expressed by a cancer cell or another cell in the cancer cell microenvironment.

In some embodiments, the composition can be for use in detecting, monitoring, and/or imaging cancer cells and/or cancer cell metastasis, migration, dispersal, and/or invasion, and/or for treating cancer in a subject.

In other embodiments, the compositions is configured for in vivo administration to a subject or ex vivo administration to biological sample of the subject.

In some embodiments, the compound further includes a detectable moiety linked to and/or complexed with the compound. The detectable moiety can include, for example, at least one of a contrast agent, imaging agent, radiolabel, semiconductor particle, or nanoparticle.

In some embodiments, the detectable moiety is detectable by at least one of magnetic resonance imaging positron emission tomography (PET) imaging, computer tomography (CT) imaging, gamma imaging, near infrared imaging, or fluorescent imaging.

In some embodiments, the compound can inhibit glioma cell migration in a scratch wound healing assay at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, the compound can inhibit aggregation of glioma sphere formation at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In still other embodiments, the compound can inhibit aggregation of PTPμ expressing SFF9 cells at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to PTPμ expressing SFF9 cells administered DMSO.

In other embodiments, the compound can inhibit PTPμ's enzymatic activity in an in vitro phosphatase assay at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In some embodiments, the compound can inhibit tumor growth at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

### BRIEF DESCRIPTION OF THE DRAWINGS

FIGS. 1(A-D) illustrate images showing the PTPμ regulatory wedge domain borders a druggable pocket. A. Structure of the PTPμ wedge domain (blue) and the D1 domain and relative position to the D2 domain (modelled after the D1 PTPμ D1 and PTPσ D2 structures, PDB IDs 1RPM and 2FH7, respectively). B. A space filling model of the residues surrounding the wedge-adjacent potential binding pocket. Y1224 is at the deepest position within the pocket. C. and D. The druggable cleft relative to the position of the wedge domain.

FIG. 2 illustrates a schematic of a functional screening approach. Seventy-four PTPμ wedge-targeted compounds and 2 blinded DMSO samples were received from Atomwise and screened at 100 μM in scratch wound healing assays using two glioma cell lines (LN229 and U87). Selected active and control compounds were taken into secondary assays (Gli36 scratch and LN229 and Gli36 sphere formation and growth assays). Inhibitors selected as being active in primary and secondary screens were tested for effects on the survival of LN229 and Sf9 cells, and selected glioma-cell inhibitors and activators were screened for effects on Sf9-PTPμ aggregation, a highly specific test for PTPμ function. These assays identified 3 high priority compounds (247678835, 247682206, 247678791) able to inhibit glioma cells and affect PTPμ-mediated aggregation. We also identified one compound able to inhibit PTPμ-mediated aggregation (247685429) that did not affect glioma cells and one compound (247685114) able to activate PTPμ-mediated aggregation and stimulate Gli36 sphere formation and growth.

FIGS. 3(A-D) illustrate a histogram and images of the LN229 scratch wound assays. A. Histogram showing the effects of all soluble wedge-targeted compounds on LN229 scratch wound closure. Cell movement into the scratches was quantified from scratch wound widths at the start and end of the assay and normalized to the average movement of cells in the unblinded DMSO control samples. Data is presented as average percentages±standard error of the means (s.e.m.), and compound bar codes are shown on the x-axis. The majority of compounds were screened with an n=2. Some priority compounds were screened with 3-6 replicates. Representative endpoint images of samples treated with DMSO (B) and two priority inhibitors (C and D) are shown. The relative migration distances for each example are indicated.

FIGS. 4(A-D) illustrate a histogram and images showing the results of the Gli36 scratch wound assays. Selected inhibitors, activators, and control compounds identified in LN229 and/or U87 scratch wound closure assays were retested at 100 μM for effects on Gli36 migration. A. Histogram showing the normalized migration distance for each treated sample. Data is presented as average % movement±s.e.m, and compound bar codes are shown on the x-axis. The majority of compounds were screened with an n of 2-4. Representative endpoint images of samples treated with DMSO (B) and two priority inhibitors (C and D) are shown. The relative migration distances for each example are shown.

FIGS. 5(A-E) illustrate a histogram and images showing the results of the LN229 sphere formation and growth assays. Selected inhibitors, activators, and control compounds identified in LN229 and/or U87 scratch wound closure assays were retested at 100 μM in a secondary assay for glioma cell (LN229) sphere formation and growth. A. Histogram showing the effects of the indicated compounds on sphere formation. On day 1, sphere footprint areas were determined and normalized to the average footprint area of the unblinded vehicle-treated controls. On day 1, a larger footprint area indicates inhibition of aggregation. B. Histogram showing the effects of the indicated compounds on sphere growth. On day 7, the changes in sphere footprint areas were calculated and normalized to the average size change of the unblinded vehicle-treated controls. On day 7, a smaller value indicates reduced growth. Growth could not be calculated for samples that fell apart on day 1 or during the assay, and this is indicated as ‘0’ growth. Data is presented as percentages±s.e.m. of 2-4 replicates. Representative images of samples treated with DMSO (C) and two priority inhibitors (D and E) are shown. Relative day 1 footprint areas and day 7 growth measurements are indicated for each example.

FIGS. 6(A-E) illustrate a histogram and images showing the results of the Gli36 sphere formation and growth assays. Selected inhibitors, activators, and control compounds identified in LN229 and/or U87 scratch wound closure assays were retested at 100 μM in a secondary assay for glioma cell (Gli36) sphere formation and growth. A. Histogram showing the effects of the indicated compounds on Day 1 sphere footprint areas. Footprint areas were measured and normalized to the average footprint area of the unblinded DMSO-treated controls. On day 1, a larger footprint area indicates inhibition of aggregation. B. Histogram showing the effects of the indicated compounds on sphere growth. Changes in sphere footprint areas were calculated and normalized to the average size change of the unblinded vehicle-treated controls. On day 7, a smaller value indicates reduced growth. For the samples that fell apart on day 1 (247677616), growth could not be calculated and this is indicated as ‘0’ growth. Data is presented as percentages±s.e.m. of 2-4 replicates. Representative images of samples treated with DMSO (C) and two priority inhibitors (D and E) are shown. Relative day 1 footprint areas and day 7 growth measurements are indicated for each example.

FIGS. 7(A-E) illustrate a histogram and images of testing the effects of selected compounds on PTPμ-dependent adhesion. Sf9 cells (which lack endogenous PTPμ) were infected with a baculovirus expressing full-length human PTPμ. Cells were harvested 48 h after infection, treated for 20 min with compounds (at 100 μM) or DMSO, and induced to aggregate by rotation. Wells were imaged as a 4×4 grid to capture the entire surface area. A. Histogram showing the effects of the selected compounds on PTPμ-dependent aggregation. Aggregates above an arbitrary footprint size (4000 μm²) were counted and normalized to the average number present in the DMSO-treated controls. Data is presented as percentages±s.e.m. of 2-6 replicates. Representative images (central frames) of samples treated with DMSO (B), two priority inhibitors (C and D), and one priority activator (E) are shown. The relative number of aggregates for each example are shown.

FIG. 8 illustrates a histogram showing the results of the phosphatase assays. Selected compounds were tested for their ability to affect the phosphatase activity of a GST-tagged PTPμ construct comprising the entire intracellular domain of human PTPμ. The enzyme was pretreated on ice for 10 min, and the reactions started by addition of a peptide substrate and incubation at 30 °C. The amount of released phosphate was measured at 15 min using the malachite green reaction and normalized to that of the vehicle-treated control. Data is presented as percentages±s.e.m. of the indicated number of independent experiments. Differences were assessed using the Student's t-test with comparison to an untreated control sample (as all DMSO samples were set to 100% release). A difference was deemed significant at p<0.05. One priority compound (247678791) caused a modest, but statistically significant, reduction on enzymatic activity.

FIGS. 9(A-C) illustrates a plot and images of the results of the human glioma tumor model in mice. LN229-flank tumors (n=6 per treatment group) established in nude mice were injected with vehicle or compound once a week for three weeks. A. Tumor sizes were measured once a week for four weeks and normalized to their starting sizes. Data is presented as average percent growth±s.e.m. Between-group comparisons were made using Student's t-test. Differences were considered significant at p<0.05. 247678835 slowed tumor growth, producing a statistically significant growth reduction by 3 weeks post-first injection; however, growth seemed to rebound once treatment was stopped, and the slowed growth rate was no longer statistically appreciable at 4 weeks post-injection. Representative images of H&E-stained sections from tumors (two per treatment group) harvested at 4-weeks are shown. Tumors treated with 247678835 appeared smaller and less cellular based on the density of nuclei.

FIGS. 10(A-G) illustrate representative endpoint images of LN229 scratch wounds treated with DMSO or selected priority compounds. A-F. Endpoint images of samples treated with DMSO or the indicated inhibitors. G. Endpoint images of a sample treated with a weak activator. The distance moved relative to controls for each example is indicated.

FIGS. 11(A-D) illustrate a histogram and images of the results of the U87 scratch wound assays. A. Histogram showing the effects of all soluble wedge pocket-targeting compounds on U87 scratch wound closure. Cell movement into the scratches was quantified from scratch wound widths at the start and end of the assay and normalized to the average movement of cells in the unblinded DMSO control samples. Data is presented as average percentages±s.e.m., and compound bar codes are shown on the x-axis. Most compounds were screened with an n of 2-4. Representative images of scratch wounds treated with DMSO (A) or two priority inhibitors (C and D) are shown.

FIGS. 12(A-G) illustrate representative endpoint images of U87 scratch wounds treated with DMSO or selected priority compounds. A-F. Endpoint images of samples treated with DMSO or the indicated inhibitors. G. Endpoint images of a sample treated with a weak activator. The distance moved relative to controls for each example is indicated.

FIG. 13 illustrates images of representative examples of compounds (100 PM) that exhibited insolubility in scratch and sphere assays.

FIGS. 14(A-G) illustrate representative images of Gli36 scratch wounds treated with the indicated priority compounds. A-F. Endpoint images of samples treated with DMSO or the indicated inhibitors. G. Endpoint images of a sample treated with a weak activator. The distance moved relative to controls for each example is indicated.

FIGS. 15(A-D) illustrate a histogram and images of titration of selected compounds on LN229 sphere formation and growth. LN229 cells were plated onto non-adherent surfaces and treated with the indicated compounds at 100, 50, and 25 μM. A. On day 1, sphere footprint areas were determined and normalized to the average footprint area of the unblinded vehicle-treated controls. On day 1, a larger foot-print area indicates inhibition of aggregation. B. On day 7, the changes in sphere footprint areas were calculated and normalized to the average size change of the unblinded vehicle-treated controls. On day 7, a smaller value indicates reduced growth. Growth could not be calculated for samples that fell apart on day 1 or during the assay, and this is indicated as ‘0’ growth. Data is presented as percentages±s.e.m. The initial test at 100 μM and the follow-up at that dose with titration is shown. Each bar is the average of 2 replicates. Representative day 1 (C) and day 7 (D) images of samples treated with two priority inhibitors are shown. Relative day 1 sphere footprint areas and day 7 growth for each example are indicated.

FIG. 16 illustrates representative images of LN229 and Gli36 spheres cultured in the presence of DMSO or the indicated priority compounds (100 μM). The relative day 1 footprint area and day 7 size change for each compound are indicated.

FIG. 17 illustrates images showing the effects of prioritized inhibitors on cell survival. LN229 cells were plated onto non-adherent surfaces and cultured in the presence of the indicated compounds (100 μM). On day 1, spheres were stained with Helix Blue to detect dying cells. Parental Sf9 cells (which lack PTPμ) plated onto tissue culture plastic were also grown in the presence of the indicated compounds and, on day 1, stained with Helix Blue. Three compounds appeared to cause a qualitative increase in staining in LN229 spheres. No compound was toxic to Sf9 cells. There is variability in the level of Helix Blue staining exhibited by LN229 control spheres, so two untreated examples are shown.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol. the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines: a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom, such as oxygen in oxo, carbonyl, carboxyl, and ester groups; and nitrogen in groups such as imines, oximes, hydrazones, and nitriles. For example, “substituted” includes any of the above groups in which one or more hydrogen atoms with —NRgRh, —NRgC(═O)Rh, —NRgC(═O)NRgRh, —NRgC(═O)ORh, —NRgSO2Rh, —OC(═O) NRgRh, —ORg, —SRg, —SORg, —SO₂Rg, —OSO₂Rg, —SO₂ORg, ═NSO₂Rg, and —SO₂NRgRh. “Substituted” also means any of the above groups in which one or more hydrogen atoms are replaced with —C(═O)Rg, —C(═O)ORg, —C(═O)NRgRh, —CH₂SO₂Rg, —CH₂SO₂NRgRh. In the foregoing, Rg and Rh are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

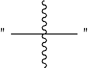

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

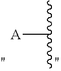

indicates that the chemical entity “A” is bonded to another chemical entity via the point of attachment bond. Furthermore, the specific point of attachment to the non-depicted chemical entity can be specified by inference. For example, the compound

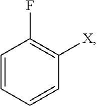

wherein X is

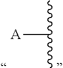

infers that the point of attachment bond is the bond by which X is depicted as being attached to the phenyl ring at the ortho position relative to fluorine.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, seminoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chondosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningioma, medulloblastoma, schwannoma and ependymoma.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Embodiments described herein relate to compounds that target receptor protein tyrosine phosphatase (RPTP) cell adhesion molecules (e.g., PTPμ), and particularly a binding pocket bordered by the wedge domain of an intracellular portion or fragment of an RPTP, and that are capable of inhibiting RPTP mediated adhesion of cells and/or cancer cell growth and/or sphere formation and/or phosphatase activity as well as to their use in methods of treating cancer in a subject in need thereof and methods of detecting, monitoring, and/or imaging cancer cells and/or cancer cell metastasis, migration, dispersal, and/or invasion in a subject.

PTPmu (PTPμ) is a member of the receptor protein tyrosine phosphatase IIb family that participates in both homophilic cell-cell adhesion and signaling. PTPmu is proteolytically downregulated in glioblastoma generating extracellular and intracellular fragments that have oncogenic activity. The intracellular fragments, in particular, are known to accumulate in the cytoplasm and nucleus where they interact with inappropriate binding partners/substrates generating signals required for glioma cell migration and growth. Compounds targeting and/or interfering with these fragments can have therapeutic potential.

We used a deep learning neural network for drug design and discovery, to screen a molecular library of several million compounds and identified candidates predicted to interact with a binding pocket bordered by the wedge domain, a known regulatory motif located within the juxtamembrane portion of the protein. These candidates were then screened in multiple cell-based assays for effects on glioma cell motility (scratch assays) and growth in 3D culture (sphere assays), and PTPmu-dependent adhesion (Sf9 aggregation). Compounds that that affected the motility of multiple glioma cell lines (LN229, U87MG, and Gli36delta5), the growth of LN229 and Gli36 spheres, PTPmu-dependent Sf9 aggregation and/or suppressed PTPmu enzymatic activity in an in vitro phosphatase assay, and/or inhibited the growth of human glioma tumors in mice can be used as a therapeutic agent to reduce cancer cell, e.g., glioma cell or glioblastoma, growth, invasion, and/or metastasis. Additionally, such compounds can be used as a targeted molecular imaging agent in brain tumor diagnosis and/or as a targeted optical imaging agent in fluorescent guided surgical resection of brain tumors.

For example, when the compound includes a detectable moiety that is directly or indirectly linked to the compound, the compound can demarcate tumor cells in tissue sections and tumor “edge” samples, suggesting that the compound can be used as a diagnostic tool for molecular imaging of metastatic, dispersive, migrating, or invading cancers or the tumor margin. Systemic introduction of compound as described herein can result in specific labeling of the tumors.

The compounds described herein can be administered systemically to a subject and readily target cancer cells associated with proteolytically cleaved intracellular fragment of the RPTP type IIb cell adhesion molecules, such as metastatic, migrating, dispersed, and/or invasive cancer cells. In some embodiments, the compounds after systemic administration can cross the blood brain barrier to define cancer cell location, distribution, metastases, dispersions, migrations, and/or invasion as well as tumor cell margins in the subject. In other embodiments, the compounds after systemic administration can inhibit and/or reduce cancer cell growth, survival, proliferation, and migration.

The compounds described herein can therefore be used in a method of inhibiting cancer cell metastasis, migration, dispersal, and/or invasion as well as in a method of treating cancer in a subject in need thereof. The methods can include administering to a subject a compound that binds to and/or complexes with a binding pocket adjacent a wedge domain of an intracellular portion or fragment of the RPTP cell adhesion molecule in the cancer cell or tumor cell microenvironment. The compound hound to and/or complexed with the binding pocket adjacent the wedge domain of the intracellular portion or fragment of RPTP cell adhesion molecule expressed by the cancer cells can inhibit and/or reduce cancer cell growth, survival, proliferation, and/or migration as well as can be detected to determine the location and/or distribution of the cancer cells in the subject.

In some embodiments, the compound can have the structure of formula (I):

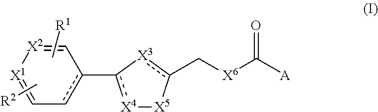

or a pharmaceutically acceptable salt, tautomer, or solvate thereof;
wherein,
a dashed line (e.g., ---- or --) is an optional bond;
A is cycloalkyl, aryl, heteroaryl, or heterocyclyl, each of which is optionally substituted with one or more R⁶.
X¹, X², and X³ are each independently C(H)ₘ or N(H)ₙ;
X⁴ and X⁵ are each independently N(H)ₙ or O;
X⁶ is CH₂ or N(R⁷);
R¹ and R² are each independently absent, ═O, halogen, hydroxyl, alkyl, haloalkyl, or alkoxy;
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocycle;
R⁷ and R⁸ are each independently H, halogen, hydroxyl, alkyl, alkoxy, or haloalkyl;
m is 0, 1, or 2;
n is 0 or 1; and
t is 0, 1, or 2.

In some embodiments, one of X¹ or X² is N(H)ₙ and the other is C(H)ₘ.

In some embodiments, one of X⁴ or X⁵ is N(H)ₙ and the other is O.

In some embodiments, X³ is N(H)ₙ.

In some embodiments, X⁶ is N(R⁷).

In some embodiments, A is:

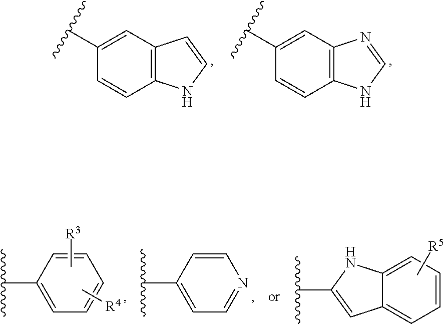

wherein,
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocycle; and
R⁵ is absent, halogen, hydroxyl, alkyl, haloalkyl, or alkoxy.

In other embodiments, the compound can have the formula selected from:

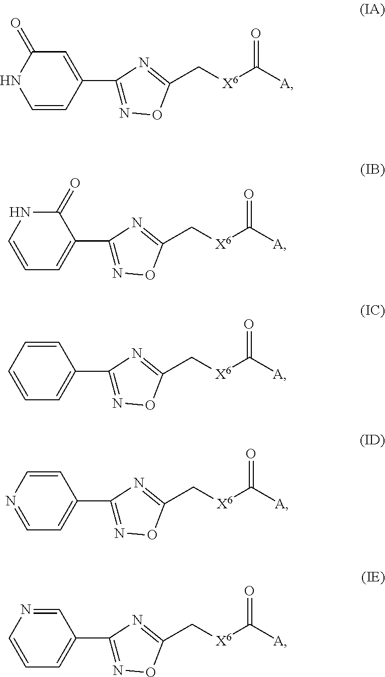

or a pharmaceutically acceptable salt, tautomer, or solvate thereof;
wherein,
A is cycloalkyl, aryl, heteroaryl, or heterocyclyl, each of which is optionally substituted with one or more R⁶;
X⁶ is CH₂ or N(R⁷);
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocycle;
R⁷ and R⁸ are each independently H, halogen, hydroxyl, alkyl, alkoxy. or haloalkyl; and
t is 0, 1, or 2.

In other embodiments, A is:

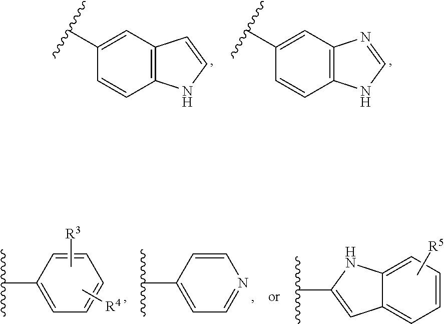

wherein,
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocycle; and
R⁵ is absent, halogen, hydroxyl, alkyl, haloalkyl, or alkoxy.

In other embodiments, the compound can have the structure of a formula selected from:

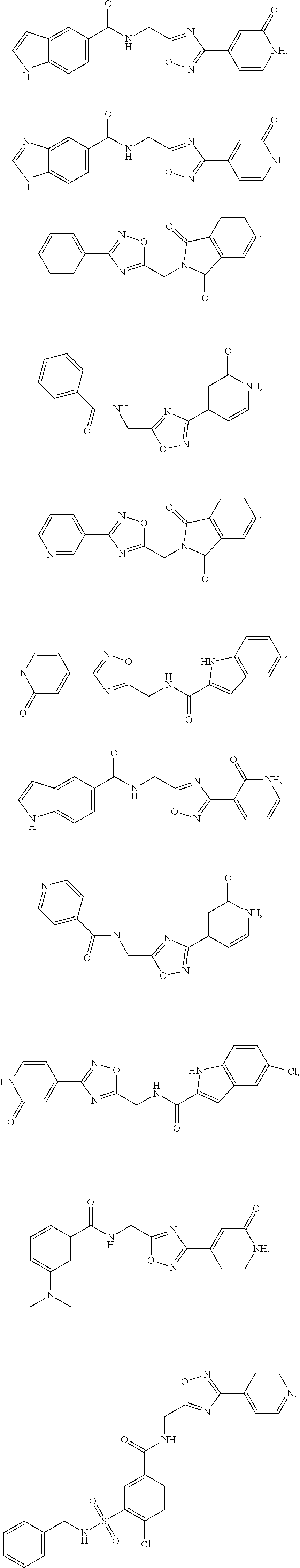

or a pharmaceutically acceptable salt, tautomer, or solvate thereof.

In other embodiments, the compound can have a structure of:

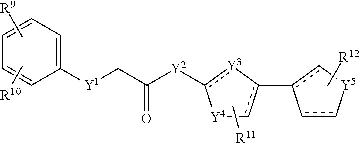

or a pharmaceutically acceptable salt, tautomer, or solvate thereof:
wherein,
a dashed line (e.g., ---- or --) is an optional bond;
Y¹, Y², Y³, Y⁴, and Y⁵ are each independently C(H)ₘ, N(H)ₙ, O, or S;
R⁹, R¹⁰, R¹¹, and R¹² are each independently absent, halogen, alkyl, hydroxyl, haloalkyl, alkoxy, —COOH, —C(O)—N(R¹³)₂, -alkylene-C(O)—N(R¹³)₂, -alkylene-OH, —C(O)O-alkyl, or -alkylene-COOH;
each R¹³ is independently H, alkyl, -alkylene-OH, —C(O)-alkyl, —C(O)O-alkyl, or -alkylene-COOH;
m is 0, 1, or 2; and
n is 0 or 1.

In other embodiments, the compound can have the formula:

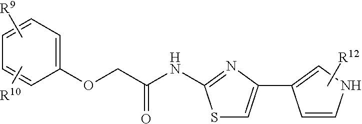

or a pharmaceutically acceptable salt, tautomer, or solvate thereof;
wherein,
R⁹, R¹⁰, and R¹² are each independently absent, halogen, alkyl, hydroxyl, haloalkyl, or alkoxy, —COOH, —C(O)—N(R¹³)₂, -alkylene-C(O)—N(R¹³)₂, -alkylene-OH, —C(O)O-alkyl, or -alkylene-COOH; and
each R¹³ is independently H, alkyl, -alkylene-OH, —C(O)-alkyl, —C(O)O-alkyl, or -alkylene-COOH.

In other embodiments, the compound can have the formula:

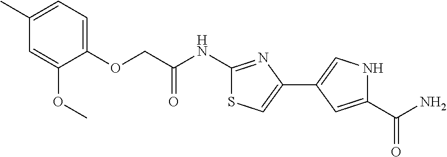 [SMILES: COc1cc(C)ccc1OCC(=O)Nc1nc(-c2c[nH]c(C(N)=O)c2)cs1]

or a pharmaceutically acceptable salt, tautomer, or solvate thereof.

In still other embodiments, the compound can be selected from:

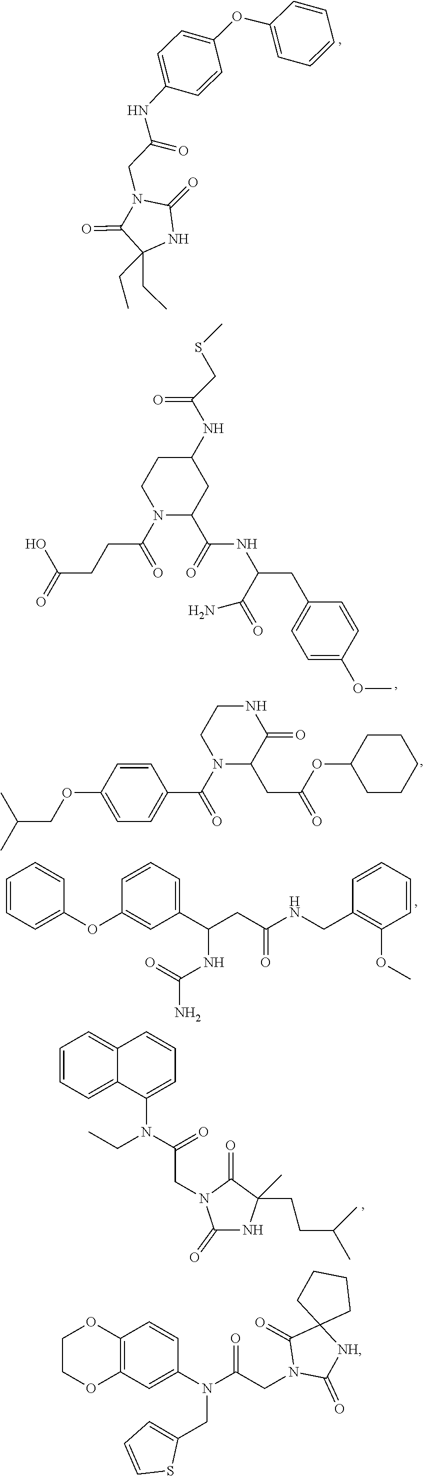

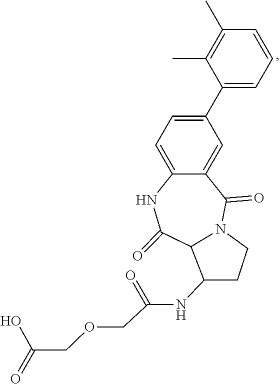 [SMILES: Cc1cccc(-c2ccc3c(c2)C(=O)N2CCC(NC(=O)COCC(=O)O)C2C(=O)N3)c1C]

or a pharmaceutically acceptable salt, tautomer, or solvate thereof.

In yet other embodiments, the compound can be selected from:

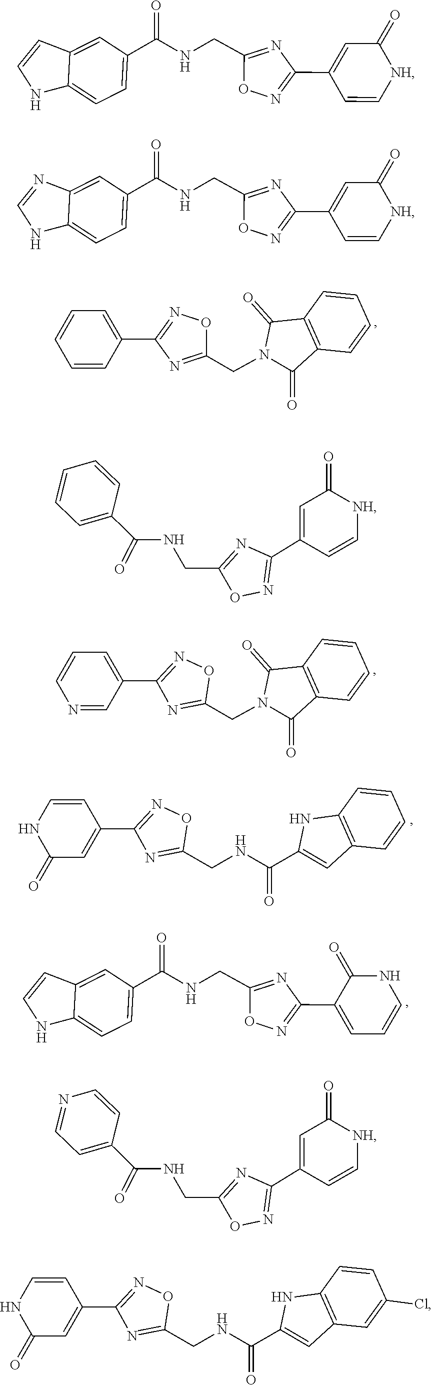

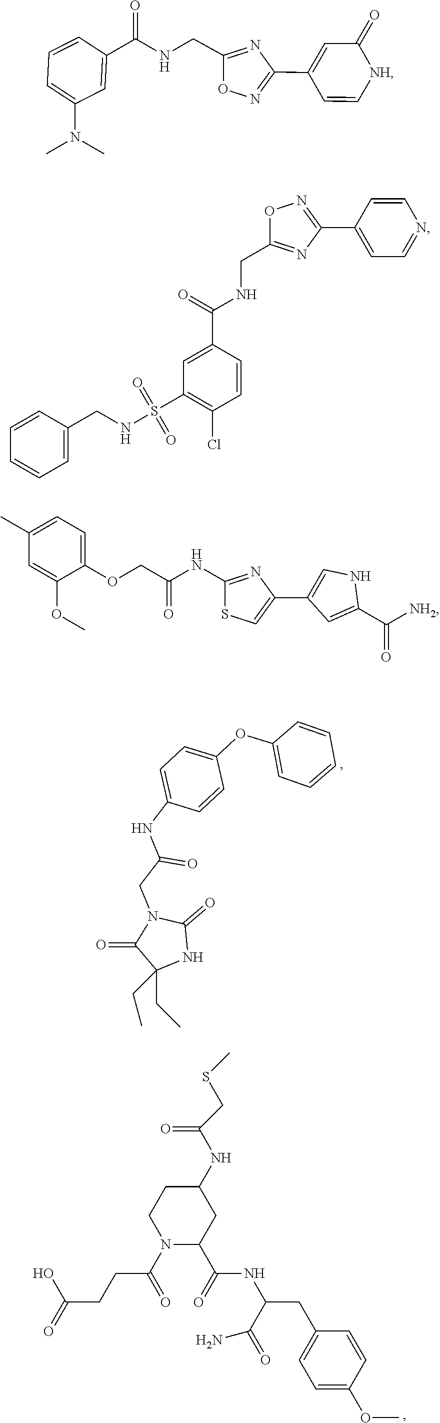

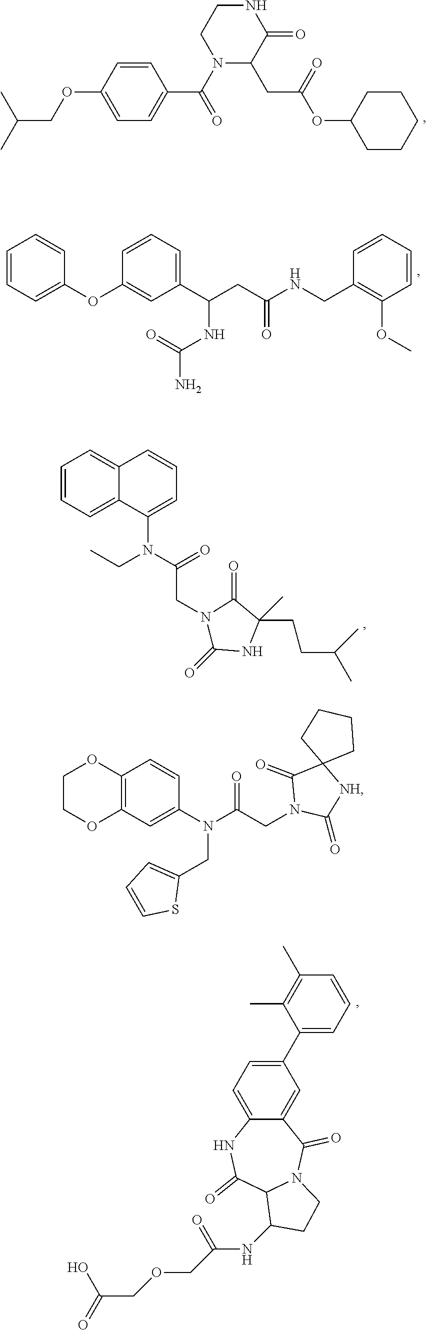

or a pharmaceutically acceptable salt, tautomer, or solvate thereof.

In some embodiments, the efficacy of the compound can be assessed by generating dose response curves from data obtained using various concentrations of a test compound. Moreover, a control assay can also be performed to provide a baseline for comparison. Such candidates can be further tested for efficacy in inhibiting chemotaxis of cancer cells in vitro, spreading, invasion, or migration of cancer cells in vitro, for efficacy in tumor dispersal, or spreading in vitro or in vivo. For example, the efficacy of the compound can be tested in vivo in animal cancer models.

Cell-based assays may be performed as either a primary screen, or as a secondary screen to confirm the activity of agents identified in a cell free screen, such as an in silico screen. Such cell based assays can employ a cell-type expressing the RPTP. Exemplary cell types include cancer cell lines, primary tumor xenografts, and glioma cells. Cells in culture are contacted with one or more compounds, and the ability of the one or more compounds to inhibit cell migration/invasion is measured. Compounds that inhibit cell migration/invasion are candidate compounds for use in the subject methods of inhibiting tumor progression. For example, the identified compounds can be tested in cancer models known in the art.

In some embodiments, putative compounds identified by in silico screens can be further screened or assessed for efficacy using scratch wound healing assays with glioma cell lines. Scratch wound healing assays measure the ability of cells to migrate into a wound and close it creating a monolayer. The scratch wound healing assays can be performed using LN229, U87, and Gli36 glioma cell lines. These cell lines express different levels of full-length PTPμ and its fragments and have different invasive behaviors in orthotopic tumor models. LN229 cells express mainly PTPμ fragments and are invasive; U87 cells express full-length and some PTPμ fragments and exhibit little invasive behavior in vivo; whereas Gli36 cells have very little full-length PTPμ but express fragments and the sensitivity profile of these cells are expected to be similar to that of the LN229 cells. Soluble wedge-targeting compounds, which are identified with an in silico screen, effects on scratch wound closure can be quantified from scratch wound widths at the start and end of the assay and normalized to the average movement of cells in the unblinded DMSO control samples.

In some embodiments, about 100 μM, preferably about 50 μM, or more preferably about 25 μM of a compound described herein can inhibit glioma cell migration in such scratch wound assays at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, efficacy of the compounds can be measured using a glioma sphere assay. The glioma cell sphere formation and growth assay tests cell-cell adhesion and the ability to grow in 3-dimensions, creating a structure that more closely mimics a tumor and its microenvironment. Compounds that are active in this assay are more likely to be effective in vivo. This assay can be selected to run in parallel with scratch wound healing assays. Glioma cells (LN229s) cultured on non-adhesive coating cluster together and grow as 3D structures that can model some of the complexity of the tumor microenvironment.

In some embodiments, about 100 μM, preferably about 50 μM, or more preferably about 25 μM of a compound described herein can inhibit aggregation of glioma sphere formation at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, efficacy of the compounds can be measured using a PTPμ-dependent Sf9 aggregation assay. The Sf9 assay directly tests adhesive action of PTPμ since Sf9 cells lack endogenous PTPμ and do not normally self-aggregate. However, baculoviral-mediated overexpression of PTPμ drives homophilic adhesion of Sf9 cells on non-adhesive coated wells. PTPμ expressing Sf9 cells readily aggregate in control samples, but wells treated with therapeutically effective compounds can contain mostly single cells or small clusters.

In some embodiments, aggregation of PTPμ expressing Sf9 cells administered about 100 μM, preferably about 50 μM, or more preferably about 25 μM, of a compound described herein can be inhibited at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to PTPμ expressing SFF9 cells administered DMSO.

In other embodiments, efficacy of compounds can be measured by the compounds' ability to alter PTPμ's enzymatic activity with an in vitro phosphatase assay. In the assay, a GST-tagged protein corresponding to the entire intracellular domain of human PTPμ can be preincubated with DMSO or selected compounds and then the reaction started by addition of a peptide substrate and incubation an elevated temperature (e.g., 30 °C). At the endpoint of the assay, released phosphate can be measured using a colorimetric reaction and normalized to the amount released by the vehicle-treated control.

In some embodiments, about 100 μM, preferably about 50 μM, or more preferably about 25 μM, of a compound described herein can inhibit enzymatic activity or released phosphate at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In other embodiments, efficacy of compounds can be measured by the compounds' ability to affect tumor growth in vivo. In such an assay, human LN229 glioma cells are subcutaneously injected into the flanks of athymic nude mice. Once tumors are established, DMSO or a test compound can be injected into the center of each tumor, and tumor volumes can calculated from caliper measurements.

In some embodiments, about 100 μM, preferably about 50 μM, or more preferably about 25 μM, of a compound described herein can inhibit tumor growth at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In other embodiments, the compounds can include or be directly or indirectly coupled to a detectable moiety. The detectable moiety can include any contrast agent or detectable label that facilitate the detection step of a diagnostic or therapeutic method by allowing visualization of the complex formed by binding of the compound to the intracellular portion or fragment of the RPTP cell adhesion molecule. The detectable moiety can be selected such that it generates a signal, which can be measured and whose intensity is related (preferably proportional) to the amount of the compound bound to the tissue being analyzed.

Any of a wide variety of detectable moieties can be linked with the compounds described herein. Examples of detectable moieties include, but are not limited to: various ligands, radionuclides, fluorescent agents and dyes, infrared and near infrared agents, chemiluminescent agents, microparticles or nanoparticles (e.g., quantum dots, nanocrystals, semiconductor particles, nanoparticles, nanobubbles, or nanochains and the like), colorimetric labels, magnetic labels, and chelating agents.

In some embodiments, compounds including the detectable moiety described herein may be used in conjunction with non-invasive imaging (e.g., neuroimaging) techniques for in vivo imaging of the compound, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a labeled compound, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of the compound along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

In one example, the detectable moiety can include a radiolabel, that is directly or indirectly linked (e.g., attached or complexed) with a compound described herein using general organic chemistry techniques. The radiolabel can be, for example, ⁶⁸Ga, ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S. Such radiolabels can be detected by PET techniques, such as described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The detectable moiety can also include ¹²³I for SPECT. The ¹²³I can be coupled to the compound by any of several techniques known to the art. In addition, the detectable moiety can include any radioactive iodine isotope, such as, but not limited to ¹³¹I, ¹²⁵I, or ¹²³I. The radioactive iodine isotopes can be coupled to the compound, for example, by conversion of a non-radioactive halogenated precursor to a stable tri-alkyl tin derivative which then can be converted to the iodo compound by several methods well known to the art.

The detectable moiety can further include known metal radiolabels, such as Technetium-99m (99mTc), ¹⁵³Gd, ¹¹¹In, ⁶⁷Ga, ²⁰¹T, ⁸²Rb, ⁶⁴Cu, ⁹⁰Y, ¹⁸⁸Rh, T(tritium), ¹⁵³Sm, ⁸⁹Sr, and ²¹¹At. Modification of the compound to introduce ligands that bind such metal ions can be effected without undue experimentation by one of ordinary skill in the radiolabeling art. The metal radiolabeled compounds can then be used to detect cancers, such as GBM in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

In some embodiments, the detectable moiety can include a chelating agent (with or without a chelated radiolabel metal group). Examples chelating agents can include those disclosed in U.S. Pat. No. 7,351,401, which is herein incorporated by reference in its entirety. In some embodiments, the chelating agent is 1,4,7,10-tetraazacyclododecane-1,4,7,10-tetraacetic acid (DOTA).

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR700, IR800, TEXAS RED, and QUASAR 670.

In some embodiments, the detectable moiety includes a fluorescent dye. Examples of fluorescent dyes include fluorescein isothiocyanate, cyanines, such as Cy5, Cy5.5 and analogs thereof (e.g., sulfo-Cyanine 5 NHS ester and Cy5.5 maleimide). See also Handbook of Fluorescent Probes and Research Chemicals, 6th Ed., Molecular Probes, Inc., Eugene Oreg, which is incorporated herein by reference.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13: 195-208; and Cancer 67: 1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor particles, can be employed as detectable moieties as described in Gao, et al “In vivo cancer targeting and imaging with semiconductor quantum dots”, Nature Biotechnology, 22, (8), 2004, 969-976, the entire teachings of which are incorporated herein by reference. The disclosed compounds can be coupled to the quantum dots, administered to a subject or a sample, and the subject/sample examined by fluorescence spectroscopy or imaging to detect the labeled compound.

In certain embodiments, a detectable moiety includes an MRI contrast agent. MRI relies upon changes in magnetic dipoles to perform detailed anatomic imaging and functional studies. MRI can employ dynamic quantitative T1 mapping as an imaging method to measure the longitudinal relaxation time, the T1 relaxation time, of protons in a magnetic field after excitation by a radiofrequency pulse. T1 relaxation times can in turn be used to calculate the concentration of a molecular probe in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed compounds can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium(III)chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, the compound can be coupled or linked to a chelating agent, such as macrocyclic chelator DOTA, and a single metal radiolabel.

The compounds described herein can be used in a pharmaceutical composition to detect and/or treat a variety of cancers that express RPTP including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma. Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoacanthoma, seminoma, thyroid follicular cancer and teratocarcinoma.

In certain embodiments, cancer cells that express an RPTP can include glioma cells. The term glioma, as used herein, refers to a type of cancer arising from glial cells in the brain or spine. Gliomas can be classified by cell type, by tumor grade, and by location. For example, ependymomas resemble ependymal cells, astrocytomas (e.g., glioblastoma multiforme) resemble astrocytes, oligodendrogliomas resemble oligodendrocytes. Also mixed gliomas, such as oligoastrocytomas may contain cells from different types of glia. Gliomas can also be classified according to whether they are above or below a membrane in the brain called the tentorium. The tentorium separates the cerebrum, above, from the cerebellum, below. A supratentorial glioma is located above the tentorium, in the cerebrum, and occurs mostly in adults whereas an infratentorial glioma is located below the tentorium, in the cerebellum, and occurs mostly in children.

In still other embodiments, the cancer cells that are detected and/or treated can include invasive, dispersive, motile or metastatic cancer cells, such as invasive, dispersive, motile or metastatic glioma cells, lung cancer cells, breast cancer cells, prostate cancer cells, and melanoma cells. It will be appreciated that other cancer cells and/or endothelial cells, which support cancer cell survival, that express an RPTP cell adhesion molecule and that can be proteolytically cleaved to produce a detectable extracellular fragment can be identified or determined by, for example, using immunoassays that detect the RPTP cell adhesion molecule expressed by the cancer cells or endothelial cells.

A pharmaceutical composition that includes a compound described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the compound probe can be by intravenous injection of the compound in the subject. Single or multiple administrations of the compound can be given. “Administered”, as used herein, means provision or delivery of compound in an amount(s) and for a period of time(s) effective to label or treat cancer cells in the subject.

In some embodiments, the compounds described herein can be administered to a cancer cell, e.g., glioblastoma multiforme cell, prostate cancer, lung cancer, melanoma, or tumor-derived endothelial cell of a subject by contacting the cell of the subject with a pharmaceutical composition described above. In one aspect, a pharmaceutical composition can be administered directly to the cell by direct injection. Alternatively, the pharmaceutical composition can be administered to the subject systematically by parenteral administration, e.g., intravenous administration or oral.

In a further example, the compound can be used in combination and adjunctive therapies for inhibiting cancer cell proliferation, growth, and motility. The phrase “combination therapy” embraces the administration of the compounds described herein and an additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). The phrase “adjunctive therapy” encompasses treatment of a subject with agents that reduce or avoid side effects associated with the combination therapy of this application.

A combination therapy is intended to embrace administration of these therapeutic agents in a sequential manner, that is, wherein different therapeutic agents are administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of therapeutic agents can be effected by an appropriate routes including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a second and different therapeutic agent) and non-drug therapies (such as, but not limited to, surgery or radiation treatment). Where the combination therapy further comprises radiation treatment, the radiation treatment may be conducted at a suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments the compounds described herein can be administered in combination at least one anti-proliferative agent selected from a chemotherapeutic agent, an antimetabolite, an antitumorigenic agent, an antimitotic agent, an antiviral agent, an antineoplastic agent, an immunotherapeutic agent, or a radiotherapeutic agent.

The phrase “anti-proliferative agent” can include agents that exert antineoplastic, chemotherapeutic, antiviral, antimitotic, antitumorigenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives. DNA topoisomerase, endostatin, epipodophyllotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

In some embodiments, a compound including or linked to a detectable can be used in a method to detect and/or determine the presence, location, and/or distribution of cancer cells, i.e., cancer cells associated with RPTP cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the compound in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the composition. In this case, the baseline and post-administration images can be compared to ascertain the presence, absence, and/or extent of a particular disease or condition.

In one aspect, the compound including the detectable moiety may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the compound can be given intravenously about 24 hours prior to pre-surgical stereotactic localization MRI. The compounds can be imaged on gradient echo MRI sequences as a contrast agent that localizes with the glioma.

Compounds described herein that include a detectable moiety and specifically bind to and/or complex with RPTP cell adhesion molecules (e.g., PTPμ) expressed by cells or cancer cells can be used in intra-operative imaging (IOI) techniques to guide surgical resection and eliminate the “educated guess” of the location of the tumor margin by the surgeon. Previous studies have determined that more extensive surgical resection improves patient survival Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Thus, compounds that function as diagnostic imaging agents have the potential to increase patient survival rates.

In some embodiments, to identify and facilitate removal of cancer cells, microscopic intra-operative imaging (IOI) techniques can be combined with systemically administered or locally administered compounds described herein. The compounds upon administration to the subject can target and detect and/or determine the presence, location, and/or distribution of cancer cells, i.e., cancer cells expressing RPTP cell adhesion molecules, in an organ or body area of a patient. In one example, the compound can be combined with IOI to identify malignant cells that have infiltrated and/or are beginning to infiltrate at a tumor brain margin. The method can be performed in real-time during brain or other surgery. The method can include local or systemic application of the compound described herein that includes a detectable moiety, e.g., a fluorescent or MRI contrast moiety. An imaging modality can then be used to detect and subsequently gather image data. The imaging modality can include one or combination of known imaging techniques capable of visualizing the compound. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, an agent including a compound linked to a fluorescent detectable moiety can be topically applied as needed during surgery to interactively guide a surgeon and/or surgical instrument to remaining abnormal cells. The compound may be applied locally in low concentration, making it unlikely that pharmacologically relevant concentrations are reached. In one example, excess material may be removed (e.g., washed off) after a period of time (e.g., incubation period).

In certain embodiments, the methods and compounds described herein can be used to measure the efficacy of a therapeutic administered to a subject for treating a metastatic, invasive, or dispersed cancer. In this embodiment, the compound can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the compound can be used to define the distribution of the metastatic cancer pre-operative and post-operative to determine the efficacy of the surgical resection. Optionally, the methods and compounds can be used in an intra-operative surgical procedure as describe above, such as a surgical tumor resection, to more readily define and/or image the cancer cell mass or volume during the surgery.

The compounds described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The compounds described herein can be administered alone as a monotherapy, or in conjunction with or in combination with one or more additional therapeutic agents. For example, the compounds described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The compound described herein and additional therapeutic agent can be components of separate pharmaceutical compositions, which can be mixed together prior to administration or administered separately. The compounds described herein, for example, be administered in a composition containing the additional therapeutic agent, and thereby, administered contemporaneously with the agent. Alternatively, the compounds described herein can be administered contemporaneously, without mixing (e.g., by delivery of the agent on the intravenous line by which the therapeutic agent is also administered, or vice versa). In another embodiment, the compounds described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The compounds described herein (or composition containing the compounds) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the compounds and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the compound in the subject, the agent can be administered between, for example, once a day or once a week.

For example, the administration of the compound and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The compounds described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of the compound described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound described herein can also depend on the disease state or condition being treated along with the clinical factors and the route of administration of the compound.

The following example is included to demonstrate preferred embodiments.

### EXAMPLE

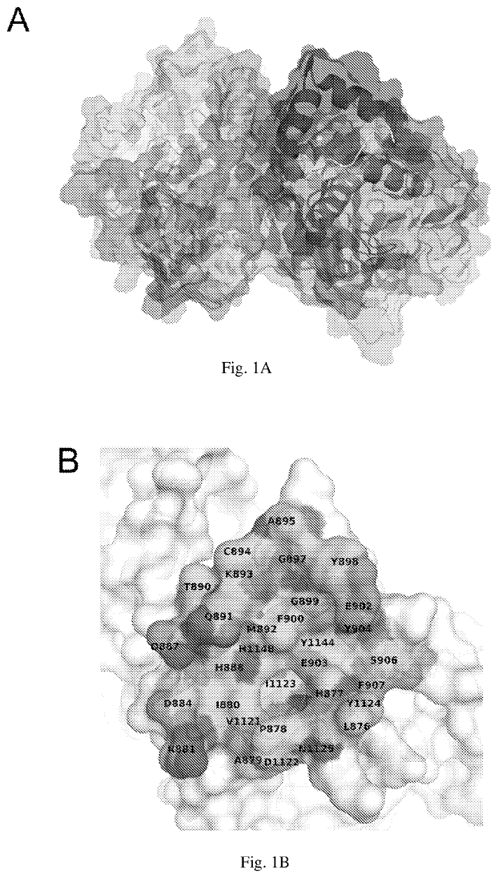

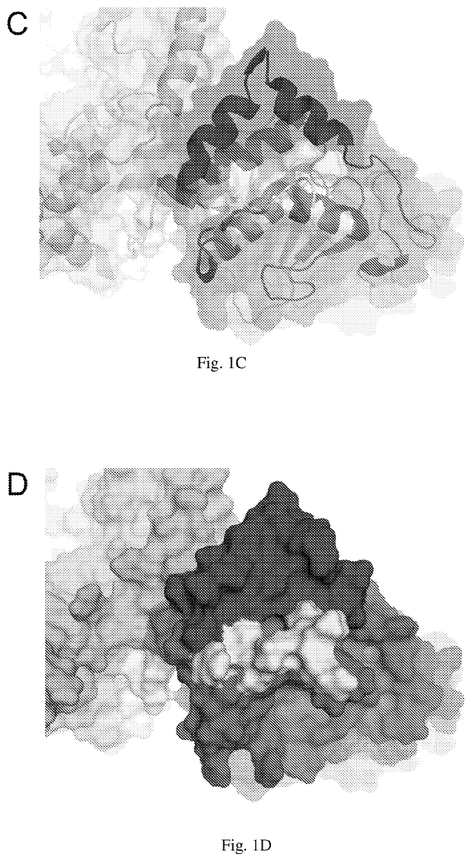

In this Example we used the AtomNet1 platform, a deep learning artificial intelligence neural network for structural based drug design. to computationally screen for small molecules predicted to interact with a binding pocket bordered by the wedge domain of PTPμ (FIG. 1) and tested these compounds in multiple cell-based assays. We identified three compounds (247678835, 247682206, 247678791) able to inhibit glioma cell migration, growth in non-adherent cultures, and, surprisingly, PTPμ-dependent adhesion. One of these compounds (247678791) was also found to modestly inhibit PTPμ's catalytic activity in vitro, and one compound (247678835), the strongest identified in the screen, was found to inhibit glioma-cell growth in a human glioma tumor model in mice. We propose that these compounds represent specific PTPμ-targeting agents that can be further developed to treat cancers including glioblastoma.

### Materials and Methods

### Compounds

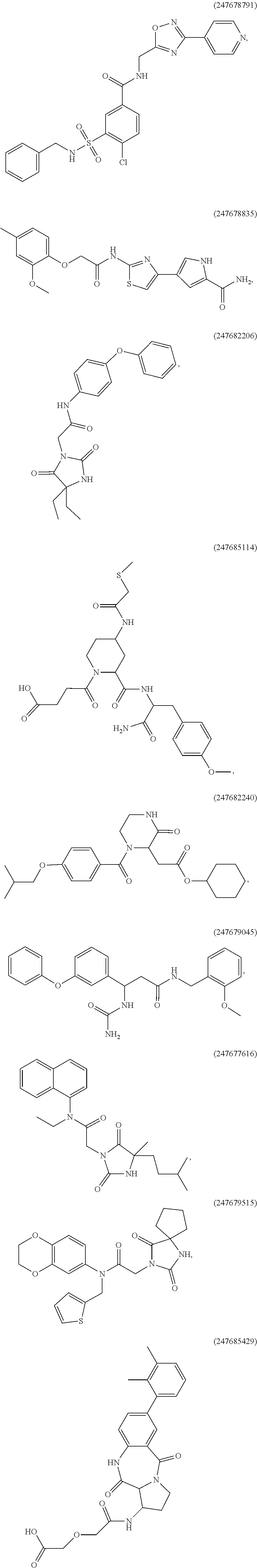

### Cell Culture

Sf9 insect cells and the human glioma cell lines LN229 (LN-229) and U87 (U-87 MG) were obtained from ATCC. The Gli36 (Gli36δ5) human glioma line was obtained from E. Chiocca and authenticated using IDEXX BioResearch (formerly RADIL: Research Animal Diagnostic Laboratory at the University of Missouri). Gli36 and U87 cells were cultured in DMEM (High Glucose DMEM, Gibco, Grand Island, NY)+10% FBS (HyClone, South Logan, UT), and LN229s were cultured in DMEM+5% FBS. All glioma cell lines were maintained at 37 °C and 5% CO₂. Sf9 cells were cultured in Grace's Complete Medium (Gibco, Grand Island, NY)+10% FBS at 27 °C.

### Scratch Wound Assays

Cells were seeded at a density of 2.7×10⁴ cells per well into the internal wells of Incuyte1 Imagelock 96-well plates (Essen BioScience Inc., Ann Arbor, MI) and cultured overnight to form monolayers. Monolayers were wounded with an IncuCyte1 96-well Woundmaker Tool per the manufacturer's instructions. The outer wells were filled with PBS to buffer edge effects, and then the wounded monolayers were cultured in 100 μl of fresh media with compounds or DMSO (2×replicates at 100 μM or 1%, respectively) at 37 °C and 5% CO₂. Images were captured every 4 hrs. using an Incucyte live cell imaging system equipped with the Scratch Wound Module. Scratch wound widths were calculated, per the manufacturer's instructions, at TO and at endpoint (typically T12 for LN229 and U87 cells and T8 for Gli36), which was taken as the last timepoint before wound closure. The cell migration distance was calculated from scratch widths [(T₀Width−T_{endpoint}Width)/2] and normalized to the average distance migrated by the DMSO controls. Values are presented as average percentages±standard errors of the means (s.e.m.). The majority of compounds were screened with an n of two, but the n for priority hits ranges from 2-6.

### Glioma Sphere Assays

Cells were seeded at a density of 7500 cells per well into the internal wells of 96-well plates coated with 0.75% (wt/vol) PVA as previously described. Compounds were added (2×replicates per treatment) at the indicated final concentrations, and control wells were treated with matching concentrations of DMSO. The external wells of the plates were filled with PBS to buffer against edge effects, and the cells were incubated at 37 °C and 5% CO₂ for 7 days. A Leica CTR6500 microscope fitted with an automated stage was used to capture brightfield images on day 1 and day 7, and sphere footprint areas were measured using Image J (v1.52a http://imagej.nih.gov/ij) as previously described. To quantify the effects of the compounds on day 1, the footprint areas of the treated wells were normalized to the average area of the matched DMSO control wells. To quantify the effects of the compounds on sphere growth, the change in the sphere footprint areas was calculated (day1/day7*100) and then normalized to the average size change of the matched DMSO samples. All values are presented as average percentages±s.e.m.

### Helix Blue Staining

LN229 cells, plated onto non-adherent surfaces as described above, and parental Sf9 cells (without PTPμ), seeded into 96-well flat bottom tissue culture plates, were treated for 24 h with the indicated compounds (100 μM). The cells and spheres were then treated with 5.5 μM Helix Blue (Biolegend, San Diego, CA) and imaged at 10× on a Leica CTR6500 fluorescence microscope.

### PTPμ-Dependent Aggregation Assay

Sf9 cells were infected with baculovirus coding for human full-length PTPμ and induced to aggregate following a modification of the procedure described in Brady-Kalnay et al. (1993). This is a new high throughput 48 well based aggregation assay for drug screening.

Briefly, 40 h after infection, cells (both floating and adherent) were gently triturated to separate clumps, and 1.14×10⁴ cells per well were seeded into 48-well culture plates pre-treated with 0.75% (wt/vol) PVA to prevent cells from adhering to the plastic. Compounds were added (2×replicates per treatment) and bubbles removed by puffing air across the plate. Each well contained a final volume of 180 μl media with compounds (at 100 μM) or DMSO (at 1%). The plates were incubated at room temperature for 20 min then rotated at 120 rpm for 30 min to induce aggregation. To facilitate automated image analysis, aggregates and loose cells, which typically swirl to the center of the wells, were distributed by manually shaking the plate before imaging the entire surface area of each well by capturing a 4×4 grid of images using a Leica CTR6500 microscope with an automated stage and a 5× objective.

### In Vitro Phosphatase Assay

A GST fusion protein containing the intracellular domain of PTPμ (B5: aa 765-1449) (PTPμ_intra) was prepared as described. Phosphatase reactions (50 μl total volume) were assembled on ice by mixing 0.4 μg GST-PTPμ_intra with phosphatase buffer (25 mM Hepes pH 7.4, 50 mM NaCl and 2 mM DTT) and compounds (100 μM) or DMSO (1%). Samples were incubated 10 min on ice and then reactions started by adding the peptide substrate to a final concentration of 60 μM and transferring the tubes to a circulating water bath at 30 °C. Reactions were stopped at 10 min by addition of malachite green dye stock (Malachite Green Phosphatase Assay Kit, Sigma-Aldrich, St. Louis, MO) prepared per the manufacturer's instructions. Colorimetric product was allowed to develop for 15 min at room temperature, and the absorbance of the samples and a standard curve of free phosphate (assembled per the manufacturer's instructions) were read at 600 nm on a Synergy HT Microplate Reader (BioTek Instruments Inc., Winooski, Vermont). The amount of released phosphate was calculated, normalized to that released by the DMSO control sample, and expressed as a percent. The data presented is the average of 3-6 independent experiments.

### Tumor Growth Assay

Glioma-cell tumor xenografts were prepared as previously described. Briefly, LN229 cells (2×10⁶ per injection) mixed with Matrigel (Corning, Corning Inc., Corning, NY, USA) were subcutaneously injected into the flank of athymic nude (FoxN1^{nu}/Foxn1^{nu}) female mice bred by the Case Western Reserve University Athymic Animal Core Facility or obtained from The Jackson Laboratory (Bar Harbor, ME). Experiments were approved by our IACUC committee. Twelve days post tumor-cell-injection, 247678835 or DMSO was diluted into PBS to give final concentrations of 2 mM or 20%, respectively, and 25 μl was injected into the center of each flank tumor. Additional compound or DMSO injections were given 7 and 14 days after the first injection, and tumor volumes [(length×width²)/2] were recorded once a week for 4 weeks. At four weeks, mice were sacrificed, and tumors were isolated, fixed in 10% buffered formalin, and prepared for paraffin sectioning. Five micron-thick sections were cut, stained with H&E, and images captured with a Hamamatsu Nanozoomer S60 Slide Scanner (Hama-matsu Photonics, K.K., Bridgewater, NJ, USA).

### Results

### Identification of Putative Small Molecule PTPμ Inhibitors Via AI-Based Virtual Screening

A small pocket on the surface of PTPμ's D1 domain (FIG. 1), close to the wedge domain, was selected for virtual screening with the AtomNet1 platform. This area sits at the interface between the D1 domain and the neighboring juxtamembrane domain for which no suitable modeling templates exist. Hence, only one half of what may be an inter-domain groove could be used for the virtual screening. The D2 domain and parts of the N-terminal linker domain were modeled after the crystal structure for the related PTPσ whereas the D1 domain was based on the available crystal structure for the PTPμ D1 domain (PDB IDs 2FH7 and 1RPM, respectively). ICM (v3.8-7 Molsoft L.L.C. San Diego, USA) was used for the homology modeling.

Atomwise used their proprietary AI screening AtomNet1 platform to screen 4 million compounds from the Mcule small-molecule library (version v20171018, https://mcule.com/) as described previously. The 2,000 top-scoring compounds were processed as follows: Compounds containing undesired (potentially reactive, unstable, or promiscuous) chemical moieties were removed. A pose filter was applied to select for compounds that are within a 4 Å heavy-atom distance from the H888 sidechain to select for those binding closely to the wedge domain and near the deepest indentation of the selected screening site. ECFP4 fingerprint-based Butina clustering using a Tanimoto coefficient of 0.4 for similarity cutoff was used to arrive at a final selection of 74 chemically diverse compounds. The selected compounds were provided as 10 mM DMSO stocks together with 2 DMSO controls as blinded samples.

### Overview of the PTPμ-Inhibitor Screen

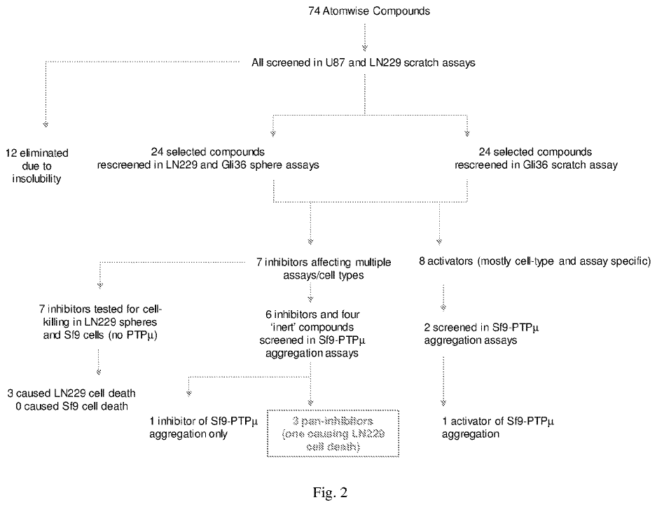

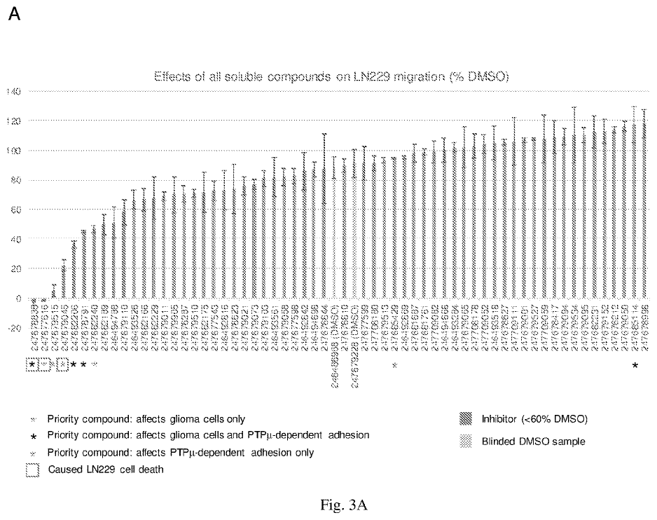

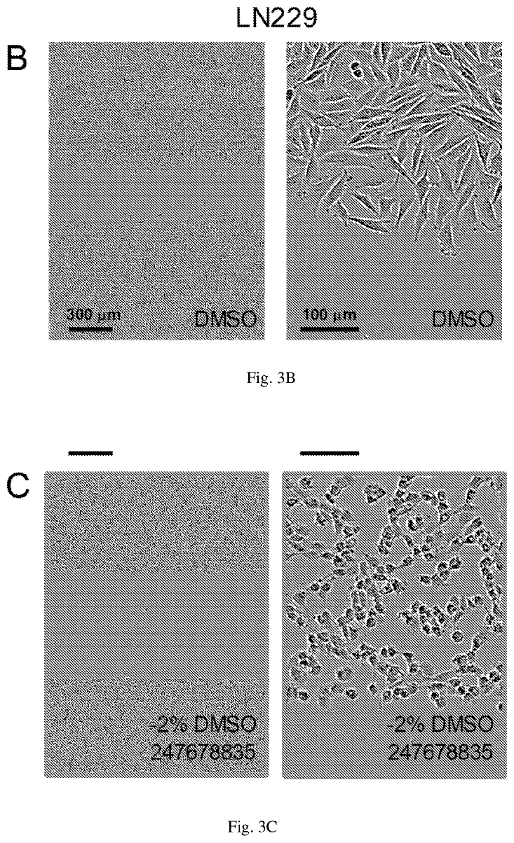

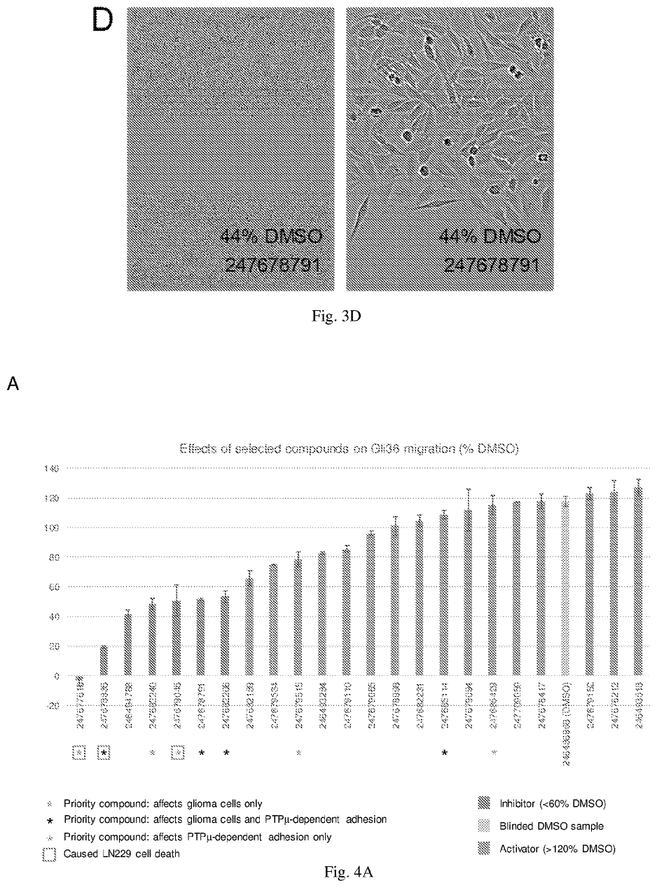

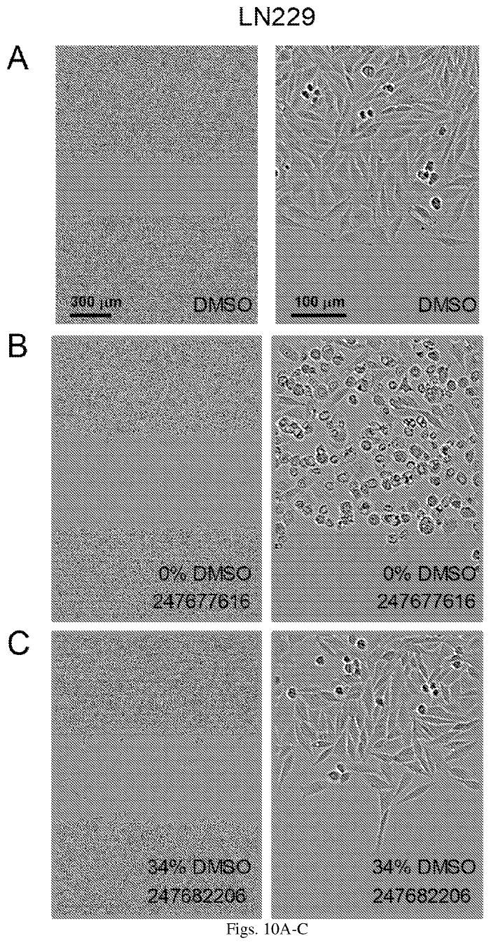

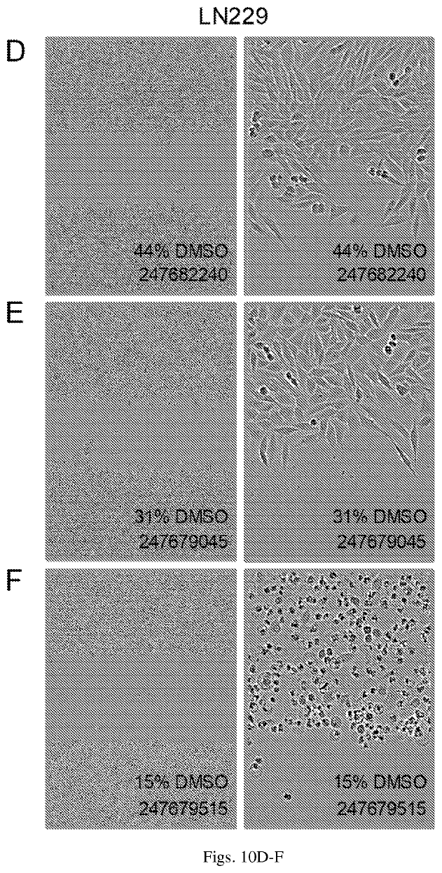

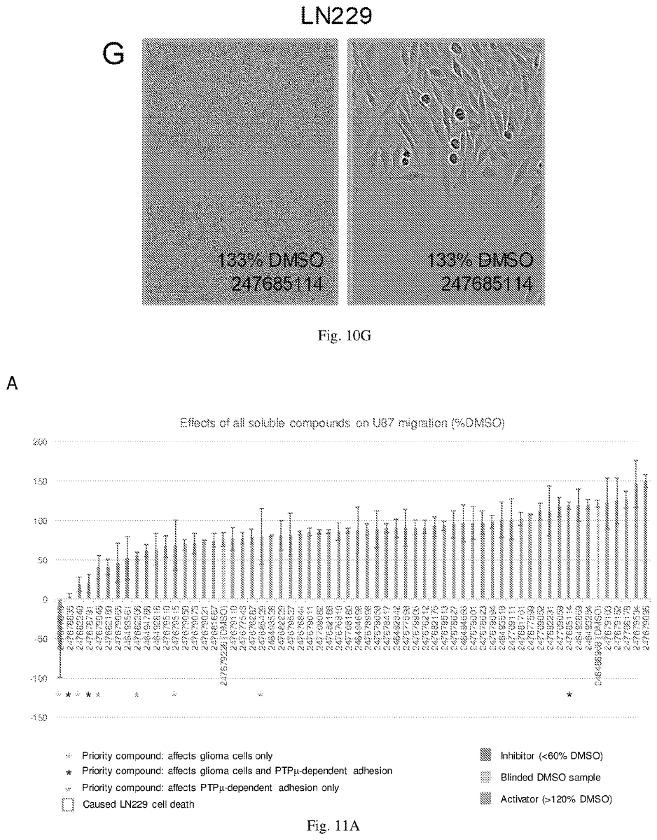

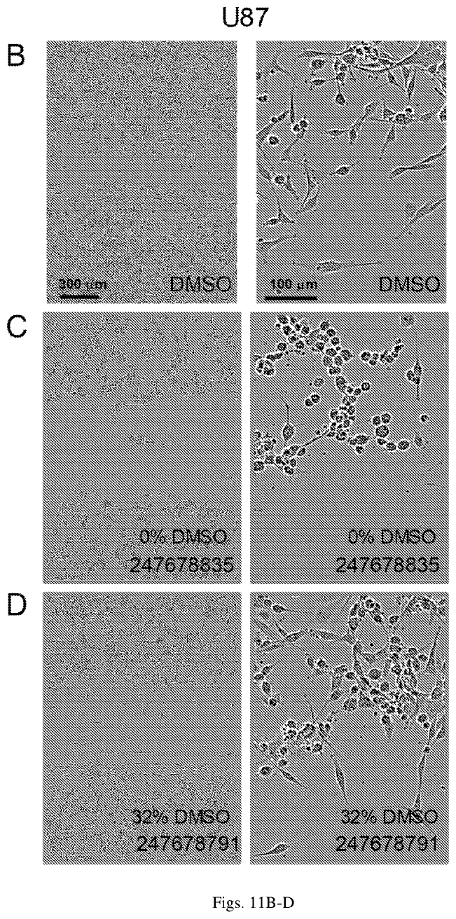

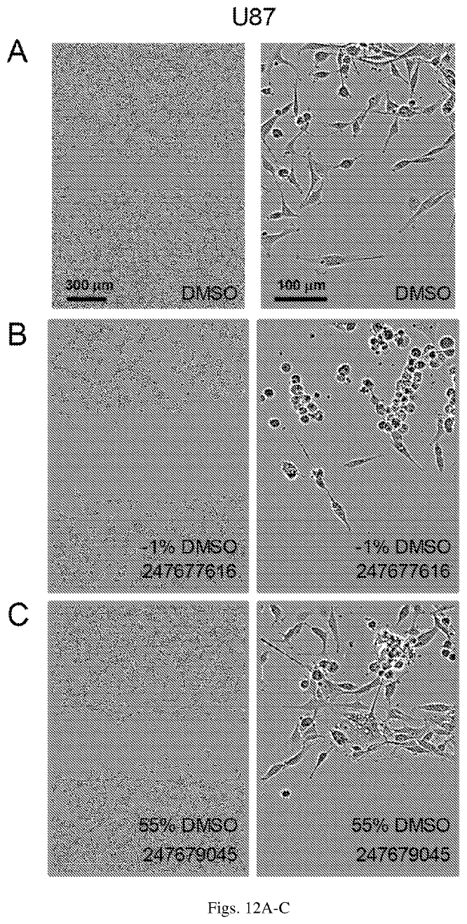

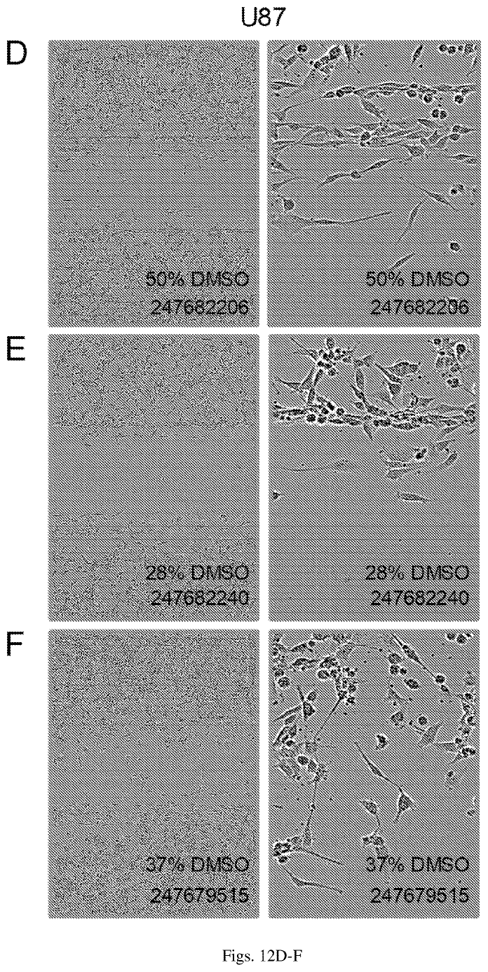

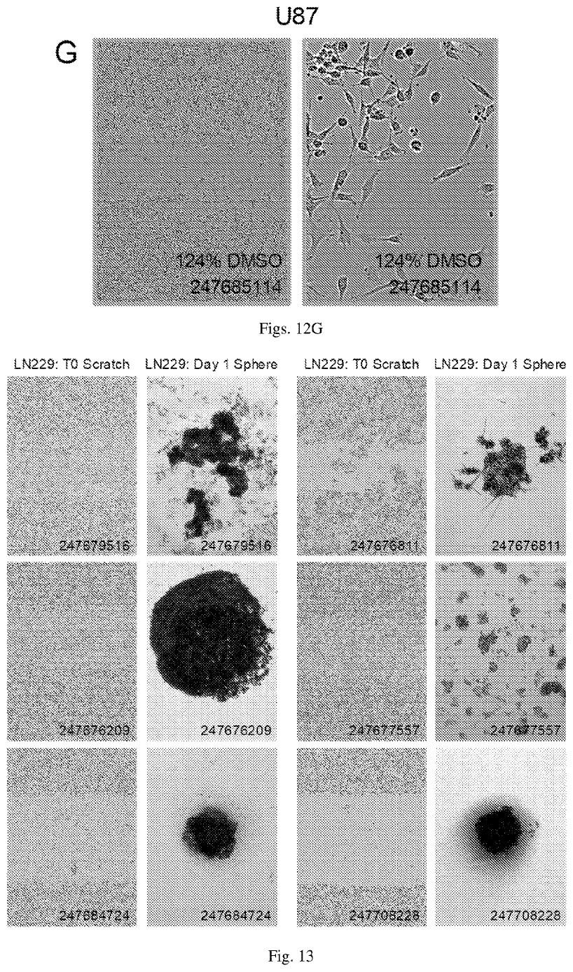

Seventy-four compounds computationally predicted to interact with the PTPμ binding pocket near the wedge domain and 2-blinded DMSO controls were received from Atomwise and screened (at 100 μM) for activity in multiple cell-based assays (FIG. 2). A non-blinded DMSO sample was used for normalization purposes in all experiments. In our primary screen, we tested the effects of the compounds on the migration of two different glioma cell lines LN229 (FIG. 3 and FIG. 10) and U87MG (U87, FIG. 11 and FIG. 12) using a scratch wound healing assay.

These cell lines were chosen because they express different levels of full-length PTPμ and its fragments and have different invasive behaviors in orthotopic tumor models. LN229 cells express mainly PTPμ fragments and are invasive; whereas, U87 cells express full-length and some PTPμ fragments and exhibit little invasive behavior in vivo. A peptide designed to target the wedge domain of PTPμ was shown to reduce LN229 migration in scratch assays by blocking the oncogenic activity of intracellular PTPμ fragments; thus, we expected compounds able to bind the wedge pocket to have a similar effect.

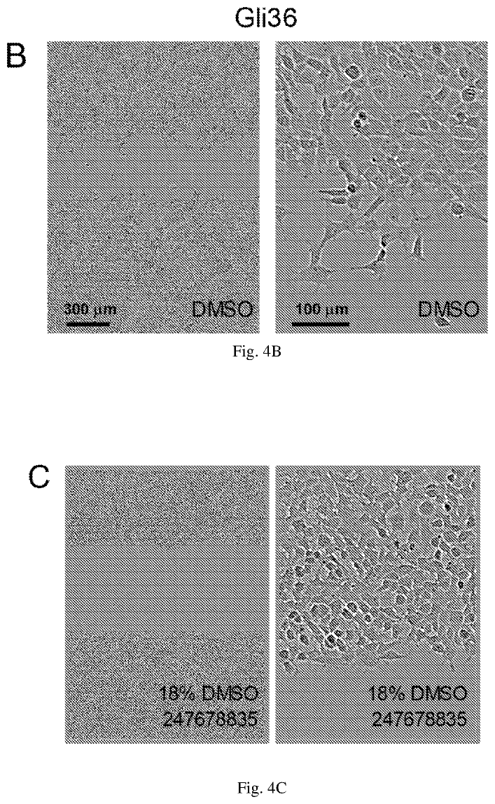

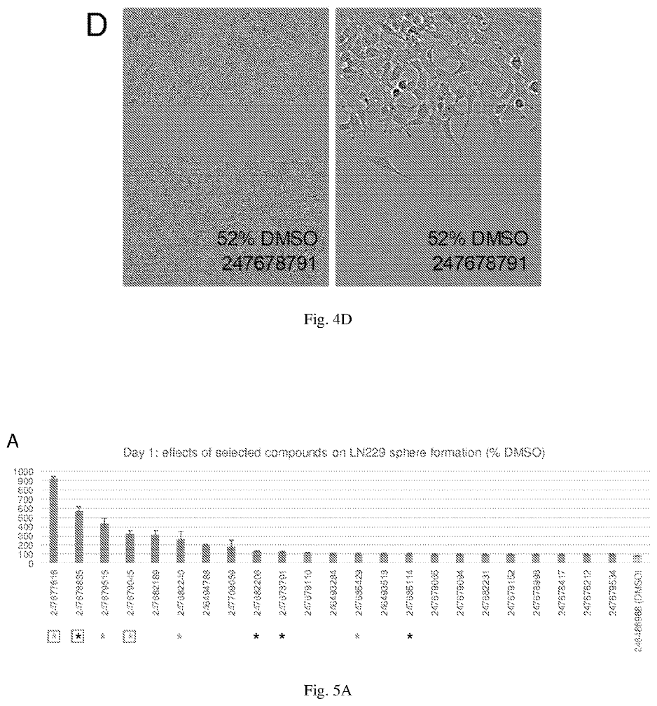

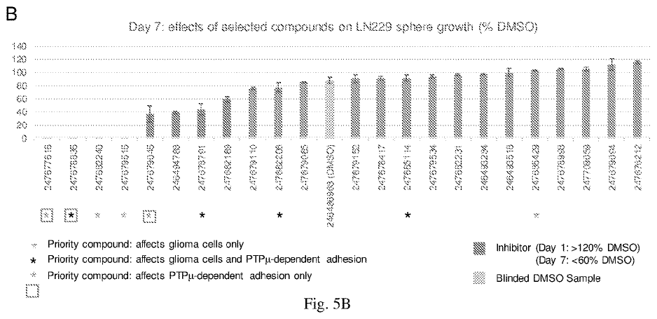

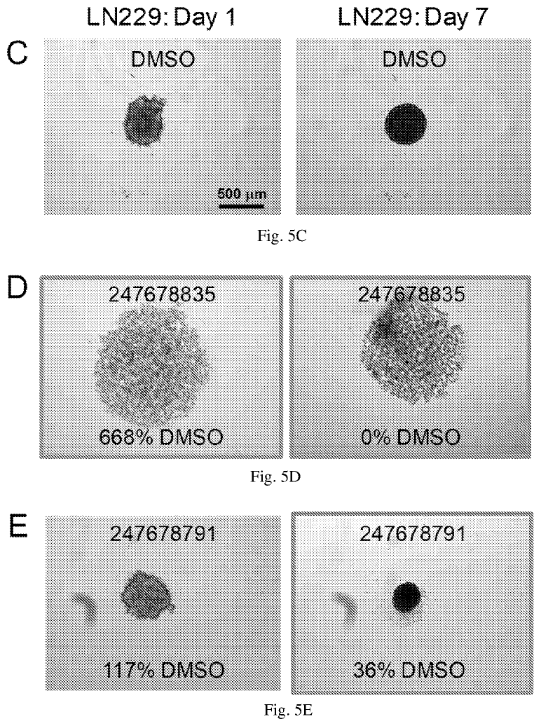

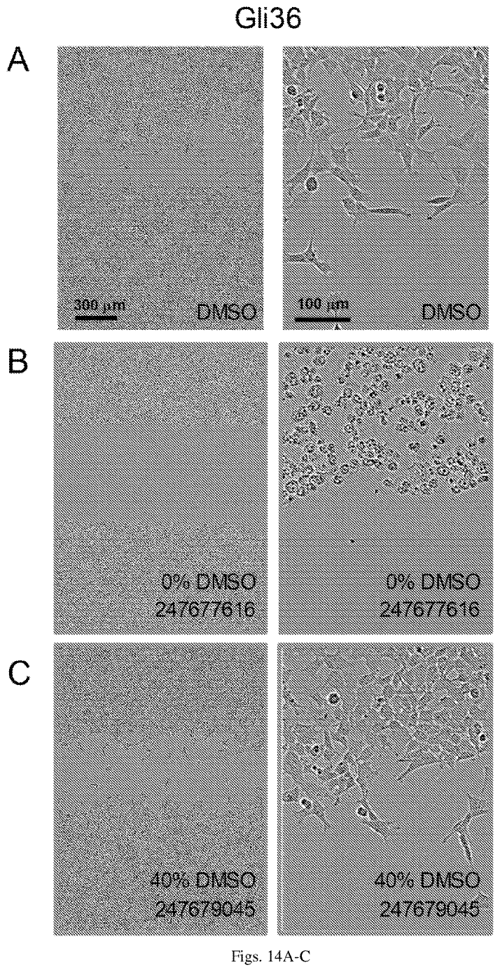

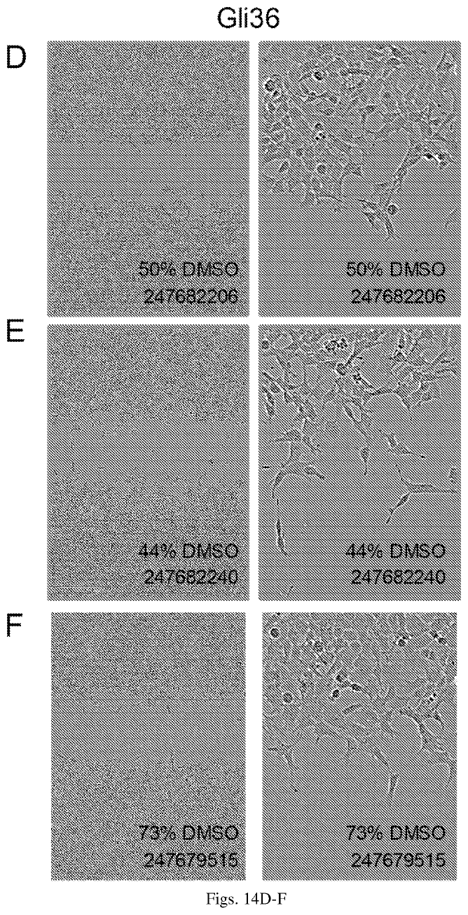

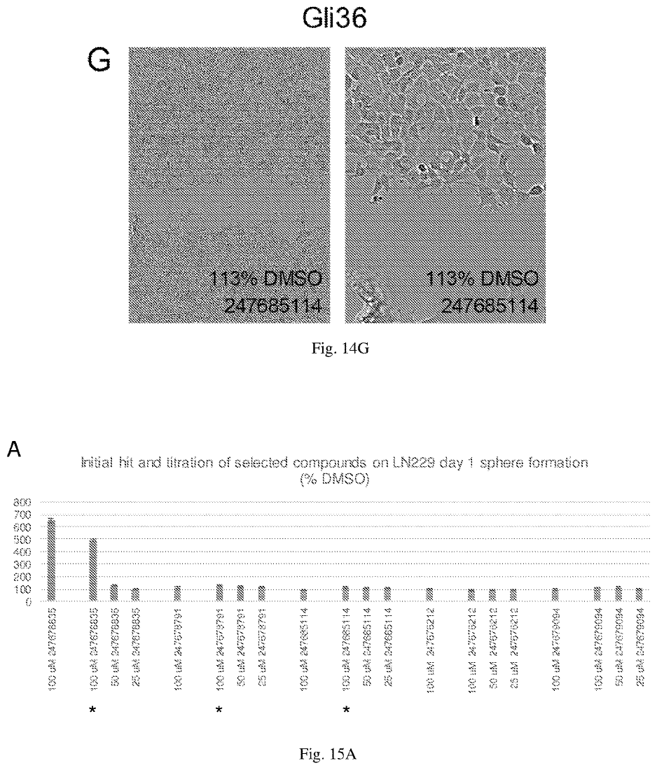

Of the 74 compounds, twelve were eliminated from the screen due to insolubility (FIG. 13), and 24 compounds (11 inhibitors of one/both cell types, four activators (all from the U87 motility screen), and nine compounds that had no effect to serve as controls) were selected for further testing on an additional cell line Gli36δ5 (Gli36) (FIG. 4 and FIG. 14) and in an additional assay, glioma cell sphere formation and growth (FIGS. 5, 6, 15 and 16). Like LN229 cells, Gli36 cells have very little full-length PTPμ but express fragments and we expected the sensitivity profile of these cells to be similar to that of the LN229 cells. The glioma cell sphere formation and growth assay was selected as a secondary screening modality as it tests cell-cell adhesion and the ability to grow in 3-dimensions, creating a structure that more closely mimics a tumor and its microenvironment. Compounds that are active in this assay are more likely to be effective in vivo.

From the primary and secondary screens, we selected seven highly penetrant inhibitors (affecting primary and secondary assays and multiple cell types) (247678835, 247677616, 247679515, 247679045, 247682206, 247678791, and 247682240). We also identified nine, mostly weak, activators of which one (247679152) affected both Gli36 and U87 migration and one (247679534) affected U87 migration and Gli36 spheres. The remaining activators were cell type/assay specific: four specifically affected U87 cell migration (246493284, 247679103, 247708178, and 247679095), two specifically affected Gli36 migration (247676212, 246493518), and one affected Gli36 spheres (247685114). Compound 247685114 was later shown to activate Sf9-PTPμ aggregation and is thus likely to be PTPμ-specific making it a high priority compound. The relevance of the other activators is unclear, and most were not considered further because they are unlikely to have therapeutic potential.

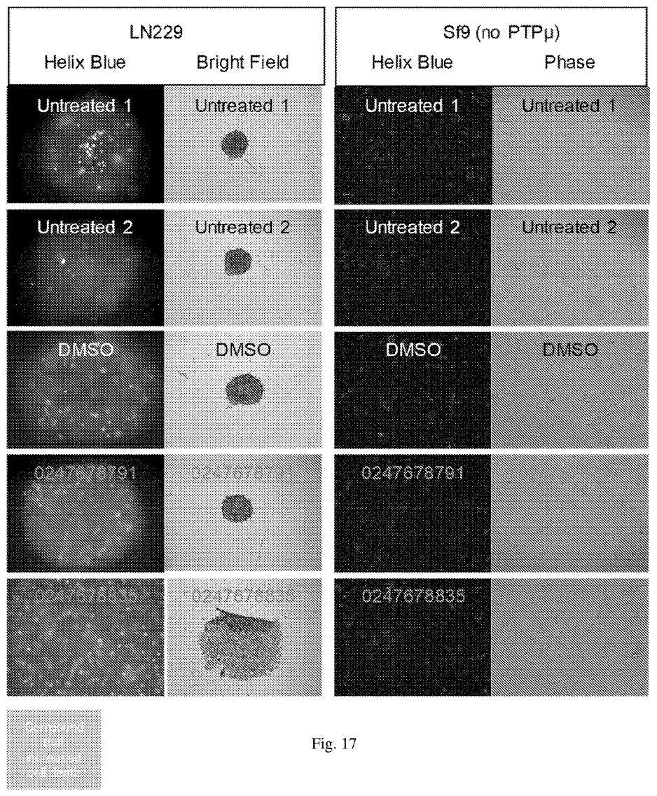

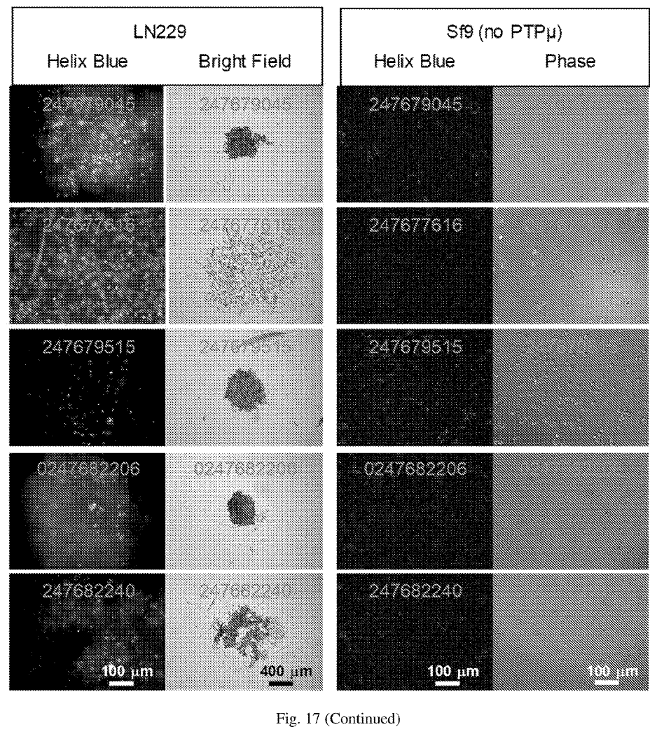

The seven penetrant inhibitors were tested to see if they affected the survival of LN229 spheres and parental Sf9 cells (which lack PTPμ) (FIG. 17). Three inhibitors (247678835, 247677616, and 247679045) caused a qualitative increase in LN229-cell death but did not affect parental Sf9 cells. This screen rules out non-specific effects on cells that do not express PTPμ. The effect on LN229 cells suggests they had a PTPμ-dependent survival effect as changes in PTPμ expression have been shown to affect cell viability.

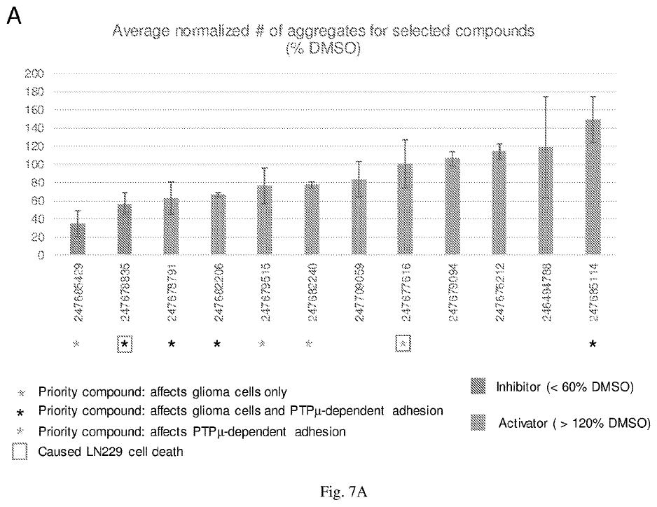

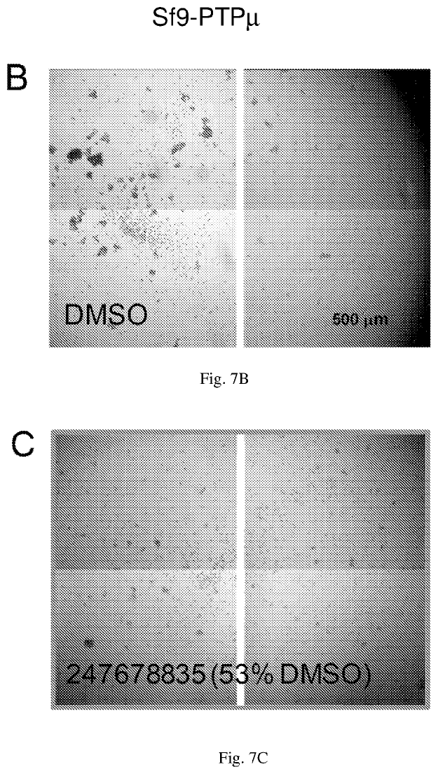

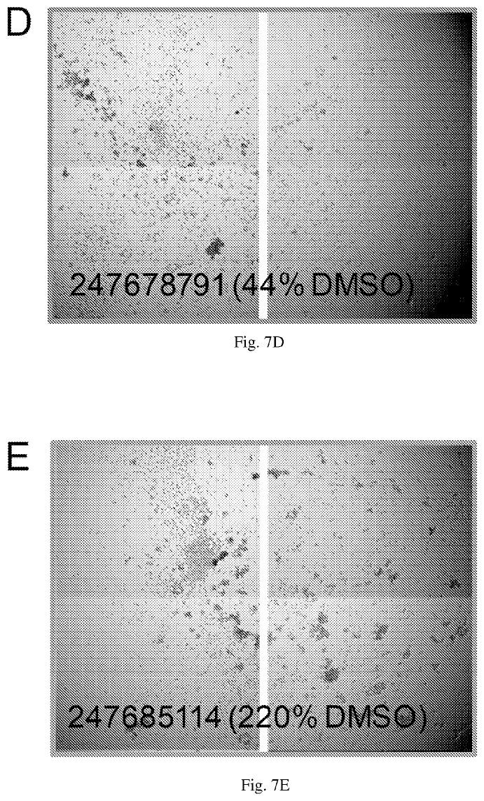

To directly test PTPμ targeting, six of the penetrant inhibitors (black and orange asterisks FIG. 7) and two activators (the Gli36 sphere-specific activator 247685114 and the strongest U87 migration-specific activator 247679095) were screened in a tertiary assay to test if the compounds can perturb PTPμ-mediated aggregation of Sf9 cells that are infected with a recombinant baculovirus to express PTPμ (Sf9-PTPμ FIG. 7). Parental Sf9 cells lack PTPμ (as well as other RPTPIIb family members), thus this assay is highly specific. The wedge domain could regulate the enzymatic activity or intracellular binding partners of PTPμ but it is unclear how this might affect PTPμ's adhesive function. Of the six tested penetrant inhibitors, only one met our strict cut-off for inhibition of Sf9-PTPμ aggregation (<60% of the average DMSO control number of aggregates). However, two additional compounds nearly reached this threshold and are also considered high priority. We also identified one inhibitor of Sf9-PTPμ aggregation that did not affect glioma cells and, curiously, one activator of PTPμ-mediated aggregation. Compound 247685114 was identified as an activator of Gli36 sphere growth and moderately stimulated LN229 (FIG. 3) and U87 cell migration (FIG. 11) (although it did not reach our strict cut-off for an activator (>120% DMSO) of cell motility).

Although, we had no expectation of identifying compounds able to perturb PTPμ's adhesive function, we regard the compounds [3 inhibitors (247678835, 247682208, and 247678791) and 1 activator (247685114)] in this category as our highest priority hits because the Sf9 assay is a short-term assay that directly tests a known function of PTPμ and is less likely to be subject to any off-target effects. To aid tracking through the various assays, these high priority com-pounds are marked by black asterisks throughout the figures (FIGS. 3-7, 11 and FIG. 15). The four penetrant glioma cell inhibitors not shown to affect PTPμ-mediated aggregation (247677616, 247679045, 247682240, and 247679515) may still have therapeutic potential and are marked by orange asterisks. The compound that inhibited PTPμ-mediated aggregation but had no effect in glioma cell assays is indicated by a blue asterisk.

### LN229, U87, and Gli36 Scratch Assays

Scratch assays measure the ability of cells to migrate into a wound and close it creating a monolayer. FIG. 3A shows the effects of all soluble wedge-targeting compounds on LN229 scratch wound closure, with the priority compounds indicated by asterisks as discussed above.

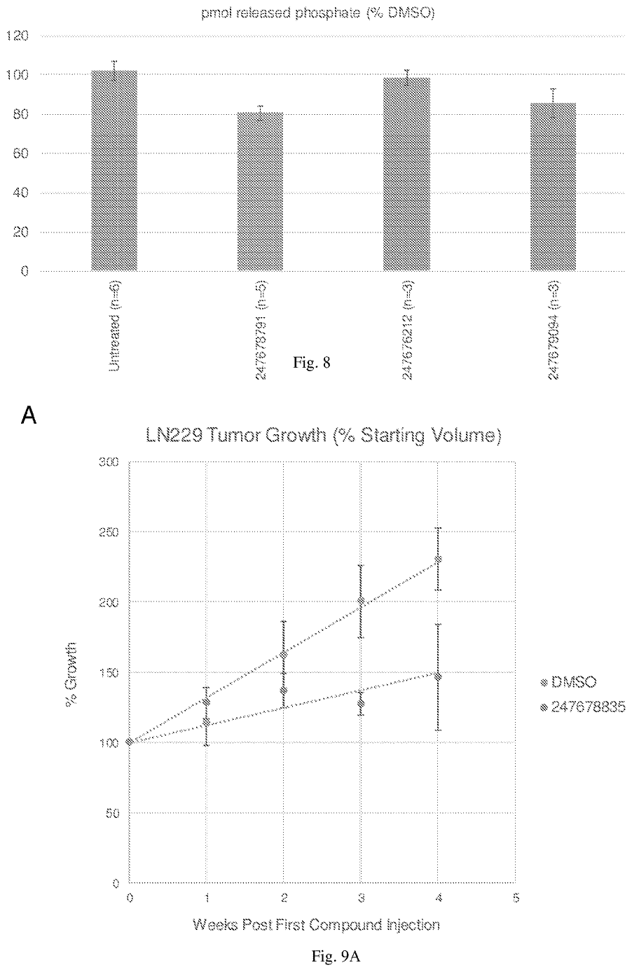

In this initial screen, we identified 10 strong LN229 inhibitors that slowed wound closure to <60% of controls (red bars) and, of these, 7 (247678835, 247677616, 247679515, 247679045, 247682206, 247678791, and 247682240) were eventually prioritized for being effective in multiple assays and on multiple cell types (FIG. 2). Also, through the screening process, we identified one priority activator (247685114) based on its ability to activate PTPμ-dependent adhesion (FIG. 8). This compound caused LN229 scratch wounds to close marginally faster (~120% of DMSO treated wounds).

There was considerable overlap between inhibitors able to affect LN229 and U87 cells (FIG. 11). However, the U87 cells seemed more ‘activatable’ than LN229s, with six compounds increasing U87 wound close rates by 20-50%. U87 cells do not form uniform monolayers, but instead grow to confluence as networks of cells connected by processes. These monolayers do not always wound cleanly and this likely accounts for the greater internal variability of the compound replicates using this cell type (see error bars in FIG. 11A). Samples with poor replicates (s.e.m.>10%) (which includes several compounds that seemed to be modestly (>120%) or strongly (>40%) activating) were not pursued further.

Representative end-point images of LN229 scratch wounds treated with DMSO (1%) or two high priority inhibitors (100 μM) are shown in FIG. 3B-3D. Compound 247678835 (which was later found to affect LN229 survival, FIG. 17) was the strongest inhibitor of LN229 wound closure and completely blocked the movement of cells into the scratch. This was accompanied by obvious morphological changes. DMSO control-treated LN229 cells were generally spindle shaped within the monolayer, but those at the wound edge had a more flattened morphology and lamellipodial ruffles consistent with being migratory (FIG. 3B). In contrast, cells treated with 247678835 were rounded with no ruffles (FIG. 3C). Compound 247678791 (which did not affect LN229 survival, FIG. 17) had subtle effects on the morphology of LN229 cells: cells at the wound edge were more spindle-shaped than flattened and exhibited fewer ruffles (FIG. 3D). Similar morphological changes were observed in U87 cells with these two priority compounds (FIG. 11C and FIG. 11D). The morphological effects of the other prioritized inhibitors on LN229 and U87 cells are shown in FIG. 10 and FIG. 12, respectively, and ranged from rounding [247677616 (FIG. 10B) and 247679515 (FIG. 10F)] to qualitatively fewer lamellipodia. Of note, some priority inhibitors [247682206 (FIG. 12D) and 247682240 (FIG. 12E)] seemed to cause a pile-up of U87 cells at the edge of the scratch wound (visible as what appears to be a chain of cells running parallel to the scratch). The priority activator did not produce obvious morphological changes in either LN229 or U87 cells. However, in the presence of this activator there did appear to be more individual U87 cells scattered within the scratch wound (FIG. 12G) consistent with the modest average increase (~20%) in the rate of U87 wound closure seen with this compound.

All inhibitors identified in the primary screens, a selection of U87-specific activators, and some apparently inert control compounds were rescreened in scratch wound assays using an additional glioma cell line (Gli36) (FIG. 4A). We found that the Gli36 cells migrated more rap-idly than LN229 and U87 cells in scratch wound assays (requiring end point images to be taken at 8 h vs. the typical 12 h timeframe of the U87 and LN229 experiments). Despite this, Gli36 cells were still sensitive to the majority of priority inhibitors, but the priority activator did not affect these cells in this assay. Three weak Gli36 activators were identified, but only one (247679152) overlapped with those previously identified in U87 cells.

FIGS. 4B-4D shows representative images of Gli36 scratch wounds treated with DMSO and two selected priority inhibitors. In vehicle-treated control samples, the Gli36 cell monolayers had a cobblestone appearance, with cells at the scratch edge extending processes and appearing to move into the scratch as interconnected chains (FIG. 4B). The strong priority inhibitor 247678835 reduced the appearance of processes and cell chains at the scratch edge (FIG. 4C), while the moderate inhibitor 247678791 did not dramatically affect Gli36 cell morphology; processes were still present and short chains of cells were seen extending into the scratch (FIG. 4D). The morphological effects of the other prioritized compounds are shown in FIG. 14. Of these, only 247677616 had a dramatic effect on Gli36 cell morphology. As seen in LN229 cells, this compound caused rounding and the appearance of intracellular phase dark areas (possibly indicating condensation/fragmentation of nuclei) (FIG. 14B). This compound was flagged as causing LN229 cell death (FIG. 17).

### LN229 and Gli36 Sphere Assays

We tested the effects of twenty-four compounds (11 flagged as inhibitory, 4 as stimulatory, and nine as inert in the initial scratch-wound screen, FIG. 2) on the ability of LN229 cells to mediate cell-cell adhesion and grow in 3D culture on non-adherent surfaces. To quantify sphere formation (FIG. 5A), the footprint areas of aggregates were measured on day 1 and normalized to that of the DMSO control. At this time point, a larger footprint size (>120%) indicates inhibition, i.e., the failure of cells to form a compact aggregate. To quantify sphere growth (FIG. 5B), we calculated the percent changes in sphere footprint areas between day 1 and day 7 and normalized them to that of the DMSO controls. At this time point, compounds that reduced growth by >40% were considered inhibitory; however, growth could not be measured for samples that fell apart on day 1 or during the culture period. Compounds that caused either effect are displayed as having 0% growth in the graph.

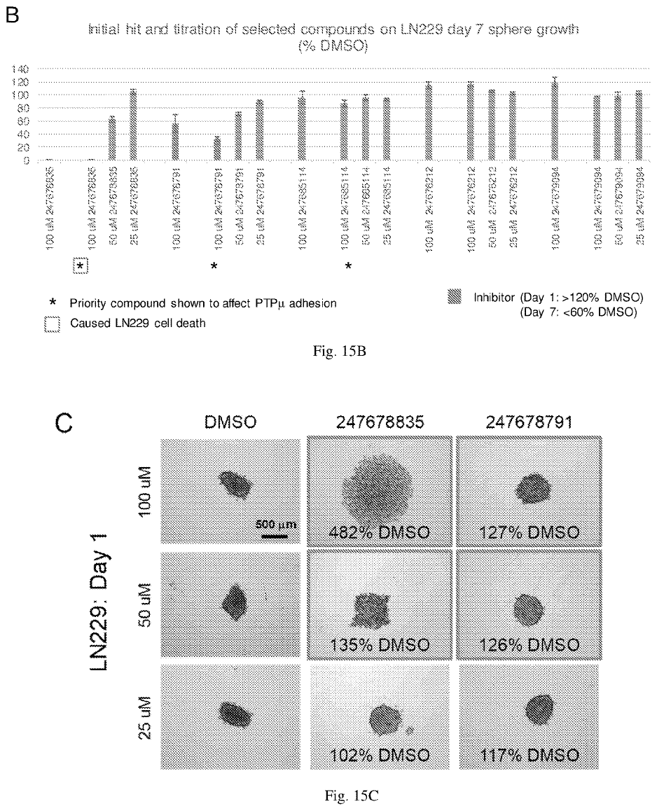

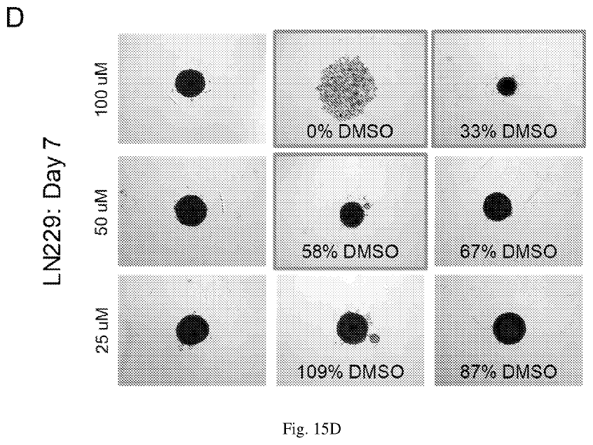

FIG. 5 shows representative images of LN229-cell aggregates cultured in the presence of DMSO (FIG. 5C) or two selected priority inhibitors (FIGS. 5D and 5E). After one day in culture, the cells treated with DMSO had formed a loose aggregate, which by day 7 had grown into a compact sphere. In contrast, the cells treated with a strong inhibitor (247678835) failed to compact and formed a mat of cells at the bottom of the well. Samples treated with a moderate inhibitor (247678791) aggregated more slowly than controls (based on the modest relative increase in footprint size measured on day 1) and grew poorly in 3D culture. These two inhibitors were tested at different dosages (25 μM, 50 μM and 100 μM) to determine the minimal dose able to affect sphere formation (FIG. 15A and C) and/or growth (FIGS. 15B and D). 247678835 dramatically disrupted sphere formation at 100 μM. This effect was still apparent at 50 μM but was less dramatic. At this dose, condensation was slowed and the resulting aggregates grew poorly in culture. 247678791 slowed aggregation at 100 and 50 μM but only reached our threshold for inhibition (<60%) of sphere growth at 100 μM.

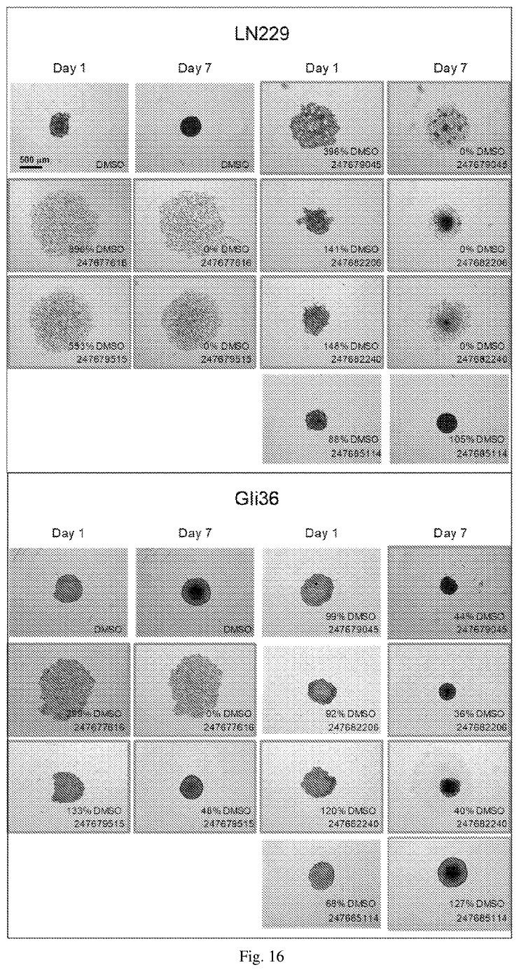

Representative images of samples treated with the other priority inhibitors (only tested at 100 μM) are shown in FIG. 16. These either completely blocked sphere formation (247677616, 247679515, 247679045) resulting in loose cells on day 1 or delayed sphere formation and inhibited growth (247682206 and 247682240), as evidenced by modestly larger footprint areas on day 1 but smaller spheres and/or loose cells on day 7. The priority activator 247685114 did not affect LN229 sphere formation or growth.

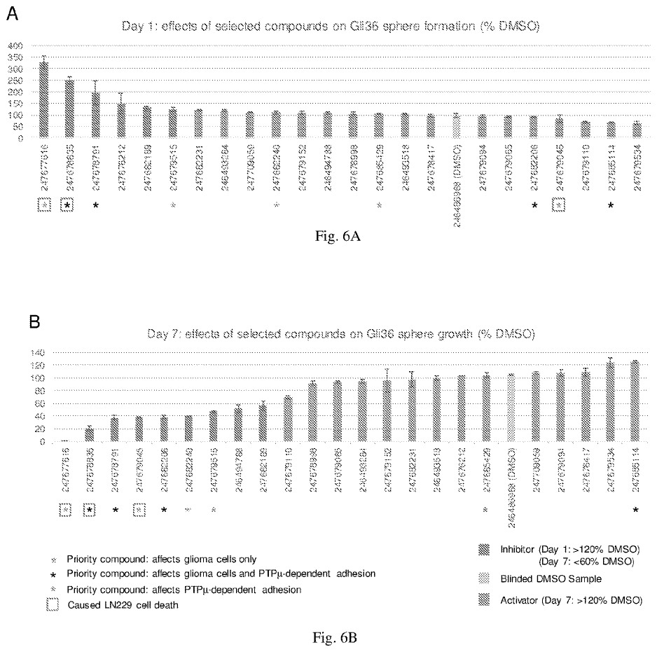

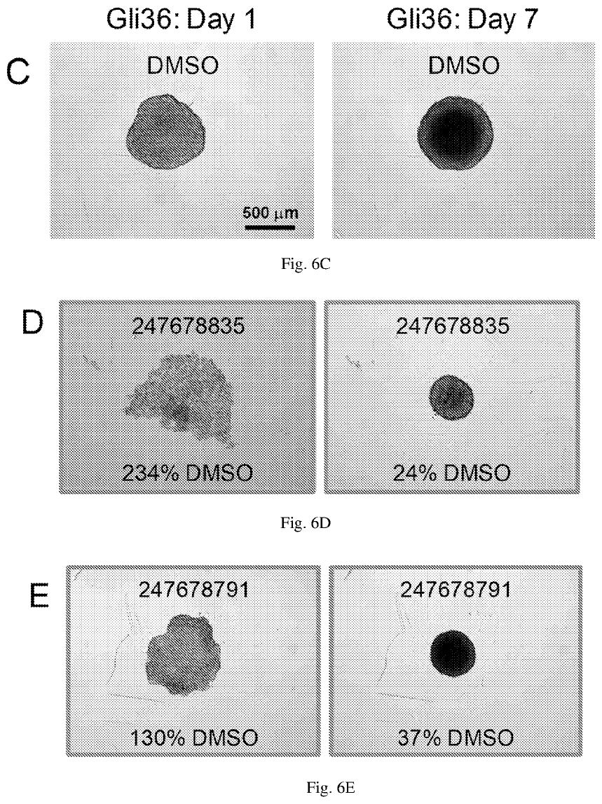

The priority inhibitors generally caused similar effects on Gli36 sphere formation and growth (i.e., slowed aggregation resulting in larger aggregates on day 1 and slowed sphere growth resulting in smaller aggregates on day 7) (FIG. 6 and FIG. 16). The strong priority compound 247678835 slowed aggregation of Gli36 cells (FIGS. 6A and 6D); however, unlike LN229 cells treated with this compound, 247678835-treated Gli36 cells still eventually formed spheres. These spheres grew poorly (FIG. 6B) and appeared more optically translucent than control spheres on Day 7 (FIG. 6D). The moderate inhibitor 247678791 also slowed condensation of Gli36 cells (FIGS. 6A and 6E) and produced spheres that grew more slowly than controls (FIGS. 6B and 6E). The effects of the other priority compounds are shown in FIG. 16. Notably, the only compound that completely blocked Gli36 sphere formation was 247677616. The priority activator (247685114) seemed to accelerate Gli36 sphere condensation and growth. The average day 1 sphere footprint area of cells treated with this compound was 68% of the control area, and these spheres grew marginally faster than controls.

### PTPμ-Dependent Aggregation Assays

Long-term cell-based assays are complex and can yield off-target effects/toxicity. We tested the effects of selected priority compounds in a short-term assay of PTPμ-dependent adhesion. Sf9 cells lack RPTPIIb family members and are not normally self-adherent but can be induced to aggregate by expressing PTPμ. This provides a highly-specific measure of PTPμ function. If the compounds had any effect on the dimerization, cis interactions, or cytoskeletal association of PTPμ they could impact PTPμ-dependent aggregation. Sf9 cells expressing PTPμ were treated with selected priority compounds (100 μM) for 20 min then induced to aggregate by rotation. The number of aggregates above an arbitrary threshold size (4000 μm²) were counted and normalized to the number present in the vehicle-treated controls (FIG. 7A). FIG. 7 shows representative endpoint images of samples treated with DMSO, 2 priority inhibitors, and one priority activator. In the DMSO-treated sample many variable-sized aggregates have formed (FIG. 7B), but samples treated with the glioma-cell inhibitors 247678835 (FIG. 7C) and 247678791 (FIG. 7D) exhibit fewer/smaller aggregates. 247685114 was flagged as a modest activator of Gli36 sphere growth (FIG. 6), but had only weak, if any, activity in other glioma-cell assays. Surprisingly, samples treated with this compound showed a considerable increase in aggregate numbers (FIG. 7E), indicating that it can stimulate PTPμ's adhesive function.

### PTPμ Enzymatic Activity

To test whether our priority compounds alter PTPμ's enzymatic activity, we used an in vitro phosphatase assay (FIG. 8). A GST-tagged protein corresponding to the entire intracellular domain of human PTPμ was preincubated on ice with DMSO or selected compounds (100 μM) and then the reaction started by addition of a peptide substrate and incubation at 30 °C. At the endpoint of the assay, released phosphate was measured using a colorimetric reaction (the malachite green assay) and normalized to the amount released by the vehicle-treated control. The data shown represents the results of 3-6 independent experiments. One of our high priority compounds (247678791) caused a modest, but highly consistent reduction in released phosphate. The strong priority compound (247678835) could not be evaluated in this assay because it reacted with the malachite green dye, giving an apparent reaction product in the absence of enzyme/substrate.

### Human Glioma Tumor Models in Mice

Our ultimate goal is to identify compounds that have therapeutic potential for treating glioblastoma. To achieve this, we need to confirm that our compounds can affect tumor growth in vivo. We chose one pan-inhibitor (247678835) to test in a human glioma xenograft flank tumor model in mice. Compound 247678835 was the strongest inhibitor identified in the initial screen of LN229 migration. It was effective in every assay and was the strongest pan-inhibitor to affect PTPμ-dependent aggregation (FIG. 7). It was also shown to be effective at inhibiting LN229 sphere growth down to 50 μM (FIG. 15), making it our primary candidate for in vivo testing.

Human LN229 glioma cells were subcutaneously injected into the flanks of twelve athymic nude mice (n=6 per treatment group). Once tumors were established (12-days post cell injection), DMSO or 247678835 was injected into the center of each tumor once a week for three weeks, and tumor volumes were calculated from caliper measurements. Individual tumor volumes were normalized to their starting volumes and the data is displayed as % growth. Mice were sacrificed 4 weeks after the first treatment, and tumors were harvested, fixed, sectioned, and stained with H&E.

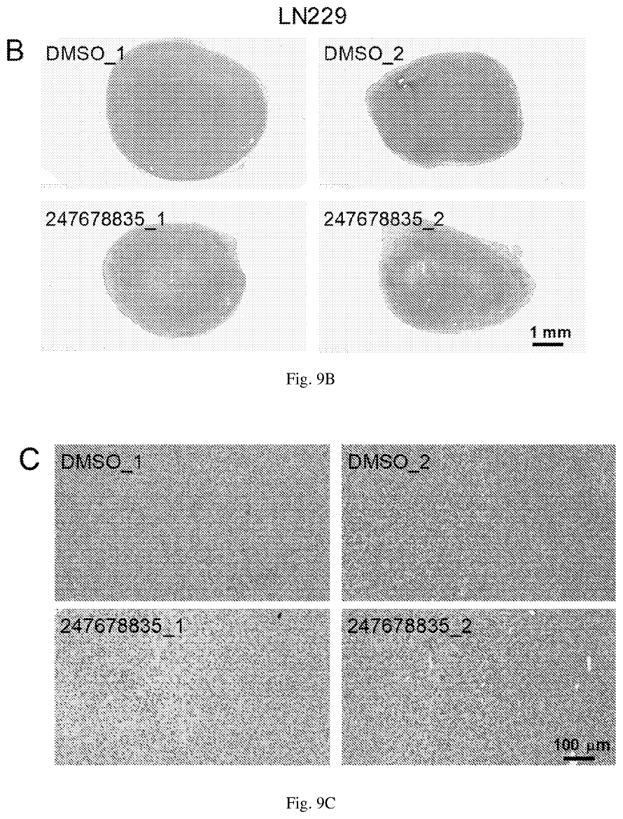

Treatment with 247678835 slowed tumor growth (FIG. 9A). At 3 weeks post first injection, DMSO-treated tumors had doubled in size, but those treated with 247678835 had only increased slightly in size (1.3×). However, by four weeks post first injection, the tumors treated with 247678835 appeared to rebound, with some resuming growth after the treatment had been discontinued (note the increase in normalized size and increased error bars). Regardless, 247678835 tumors harvested at 4 weeks generally appeared less ‘cellular’ than DMSO-treated controls based on the density of nuclei in H&E stained sections (FIGS. 9B and 9C). In fact, 247678835 induced LN229-cell death in vitro (FIG. 17).

Through AI-based computational and functional screens we identified three high priority wedge-targeting compounds that inhibit PTPμ-dependent adhesion, glioma cell migration, and glioma sphere formation and growth with the results summarized in FIG. 2. One of these compounds (247678791) was also shown to modestly inhibit the phosphatase activity of a PTPμ-intracellular construct, demonstrating a direct effect on PTPμ. Unfortunately, the strongest priority compound (247678835) could not be evaluated in our in vitro phosphatase assay system because it interacted with the dye used to measure the release of free phosphate. This compound was, however, shown to inhibit flank tumor growth in vivo, a necessary first step towards identifying compounds with therapeutic potential. Compound 247678835 was found to affect the survival of LN229 cells (which express PTPμ fragments) but not parental Sf9 cells (which lack PTPμ). We hypothesize that this compound might inhibit PTPμ fragment-dependent survival or migration signals.

We identified three additional interesting categories of compounds in this screen. We identified one compound (247685429) which was able to inhibit PTPμ-dependent adhesion but did not affect glioma cells. It is possible this compound can affect full-length PTPμ, perhaps by interfering with binding partners at the membrane, but not PTPμ fragments. Conversely, we identified four compounds (247677616, 247679515, 247679045 and 247682189) that were highly penetrant inhibitors in glioma cell assays but did not affect PTPμ-dependent adhesion. Compounds in this category could affect PTPμ fragments but not impinge on the activity of the full-length protein at the membrane perhaps by affecting binding partners. Regardless of mechanism, the inhibitory effects of these compounds on glioma cells means they are of therapeutic interest. Finally, we identified one activator of PTPμ-dependent adhesion that was weakly stimulatory in U87 and LN229 scratch assays and Gli36 sphere formation and growth assays (247685114). This was surprising, considering that restoring full-length PTPμ (and pre-sumably PTPμ-dependent adhesion) to LN229 cells was shown to inhibit their motility. However, a slight stabilization of PTPμ may stimulate contact-dependent chain cell migration. Alternatively, the compound that facilitates PTPμ-dependent adhesion in Sf9-PTPμ cells could interact in a stimulatory way with fragments in glioma cells.

The complexity of cell-based assays (which in this case involved several cell lines with differing ratios of PTPμ and full-length protein) makes it challenging to interpret the different behaviors of our compounds. However, we chose these assays over biochemical screening strategies for the following reasons: 1) Although an isolated wedge domain has been shown to mediate self-association in in vitro binding assays, it is unknown whether this happens in the context of either the full-length protein or its intracellular fragments; 2) The function of the wedge domain has been best characterized in cell-based assays, including the LN229 scratch assay, which was the basis of our primary screen; 3) Biochemical screening efforts to identify phosphatase inhibitors have limitations because compounds demonstrated to be effective in vitro often fail in vivo, in part due to membrane-permeability issues; 4) isolated assay systems cannot recapitulate all possible binding interactions necessary to reveal wedge-dependent effects. In fact, we had no a priori expectation that our screen would reveal com-pounds able to directly inhibit phosphatase activity or affect PTPμ-dependent adhesion. Constructs lacking the wedge domain induce aggregation, and, although the juxtamembrane region was found to be required for PTPμ enzymatic activity, this was not precisely mapped to the wedge domain.

We can only hypothesize how the compounds identified in this screen might affect PTPμ's functions. In regards to PTPμ-dependent adhesion, dimerization/oligomerization in the plane of the membrane is involved in stabilization of adhesion, and if the wedge domain participates in intermolecular interactions between PTPμ molecules, interfering with this might inhibit PTPμ's adhesive activity. Consistent with this, wedge peptides have been shown to self-associate, suggesting they could mediate trans interactions between PTPμ molecules. Considering enzymatic activity, the predicted compound binding pocket is approximately 20 Å away from the catalytic domain. This distance is similar to that of allosteric binding pockets identified in other phosphatase family members that are predicted to act by altering the flexibility of structures surrounding the active site that are necessary for catalysis.

In conclusion, we have identified small molecules predicted to interact with a pocket adjacent to the wedge domain of PTPμ that inhibit glioma cell migration, growth in 3D culture, PTPμ-dependent adhesion, and for one compound (247678791), phosphatase activity. Future directions will focus on direct binding assays to confirm whether/how these compounds interact with PTPμ and how this might affect downstream pathways important for glioma cell motility, survival, and/or growth. Structure activity relationship studies are also needed to optimize lead compounds. Finally, although we have preliminary evidence that one compound (247678835) was able to affect tumor growth in vivo, this was done with direct injection into the tumor.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A method of detecting, monitoring, and/or imaging cancer cells and/or cancer cell metastasis, migration, dispersal, and/or invasion, and/or treating cancer in a subject in need thereof, the method comprising: administering to the subject a compound of formula (I): 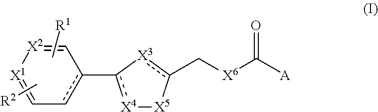
or a pharmaceutically acceptable salt, tautomer, or solvate thereof;
wherein,
a dashed line is an optional bond;
A is cycloalkyl, aryl, heteroaryl, or heterocyclyl, each of which is optionally substituted with one or more R⁶;
X¹, X², and X³ are each independently C(H)ₘ or N(H)ₙ;
X⁴ and X⁵ are each independently N or O;
X⁶ is CH₂ or N(R⁷);
R¹ and R² are each independently absent, ═O, halogen, hydroxyl, alkyl, haloalkyl, or alkoxy;
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocyclyl;
R⁷ and R⁸ are each independently H, halogen, hydroxyl, alkyl, alkoxy, or haloalkyl;
m is 0, 1, or 2;
n is 0 or 1; and
t is 0, 1, or 2.

2: The method of claim 1, wherein one of X¹ or X² is N(H)ₙ and the other is C(H)ₘ.

3: The method of claim 1, wherein one of X⁴ or X⁵ is N(H)ₙ and the other is O.

4: The method of claim 1, wherein X³ is N(H)ₙ.

5: The method of claim 1, wherein X⁶ is N(R⁷).

6: The method of claim 1, wherein A is: 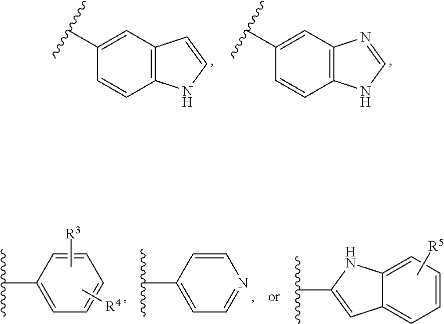
wherein,
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocyclyl; and
R⁵ is absent, halogen, hydroxyl, alkyl, haloalkyl, or alkoxy.

7: A method of detecting, monitoring, and/or imaging cancer cells and/or cancer cell metastasis, migration, dispersal, and/or invasion, and/or treating cancer in a subject in need thereof, the method comprising: administering to the subject a compound selected from: 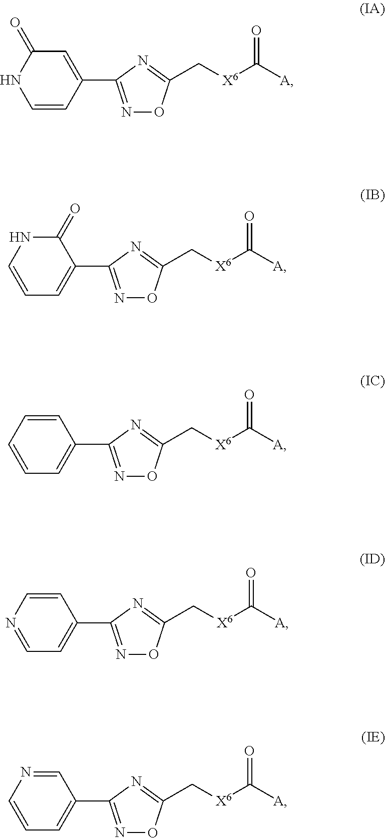
or a pharmaceutically acceptable salt, tautomer, or solvate thereof;
wherein,
A is cycloalkyl, aryl, heteroaryl, or heterocyclyl, each of which is optionally substituted with one or more R⁶;
X⁶ is CH₂ or N(R⁷)
each R⁶ is independently —N(R⁸)₂, halogen, hydroxyl, alkyl, haloalkyl, alkoxy, —S(O)ₜN(H)-alkylene-aryl or alternatively R⁶ and R⁷ together with the atom(s) to which they are attached can form a 4- to 7-membered heteroaryl or heterocyclyl;
R⁷ and R⁸ are each independently H, halogen, hydroxyl, alkyl, alkoxy, or haloalkyl; and
t is 0, 1, or 2.

8: The method of claim 7, wherein A is: 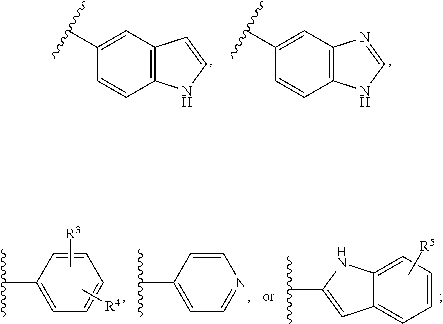
R³ and R⁴ are each independently absent, —N(R⁸)₂, halogen, hydroxyl, alkyl, haloalkyl, alkoxy, —S(O)ₜN(H)-alkylene-aryl or alternatively R³ or R⁴ and R⁷ together with the atom(s) to which they are attached can form a 4- to 7-membered heteroaryl or heterocyclyl; and
R⁵ is absent, halogen, hydroxyl, alkyl, haloalkyl, or alkoxy.

9: The method of claim 7, wherein X⁶ is N(R⁷).

10: The method of claim 1, wherein the compound has a formula selected from: 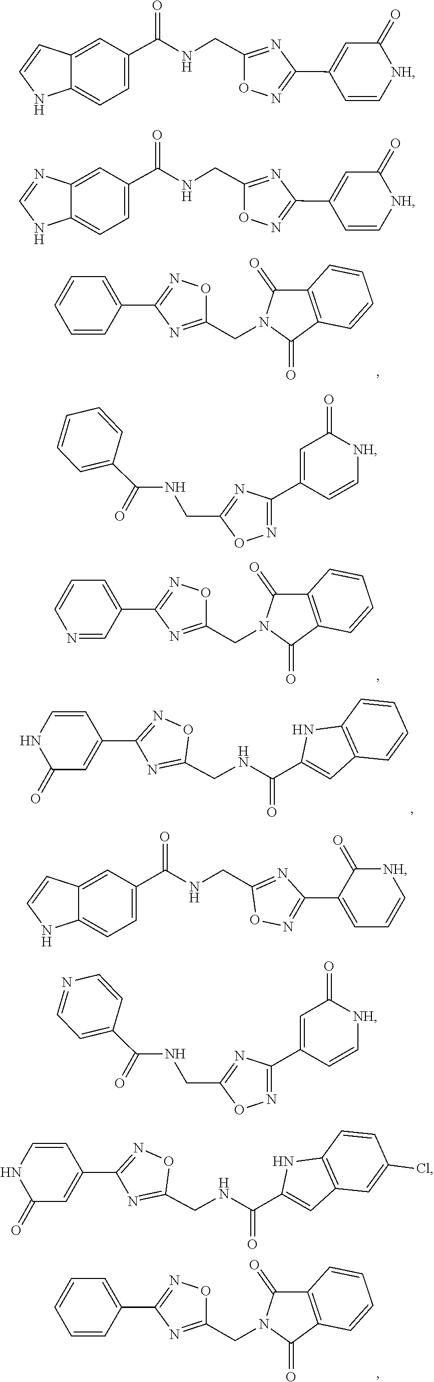 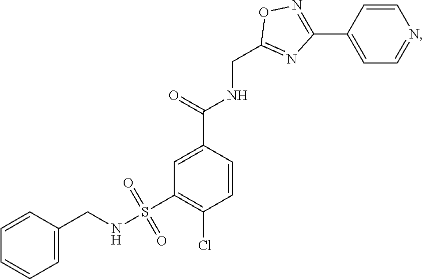 [SMILES: O=C(NCc1nc(-c2ccncc2)no1)c1ccc(Cl)c(S(=O)(=O)NCc2ccccc2)c1]
or a pharmaceutically acceptable salt, tautomer, or solvate thereof.

11-15. (canceled)

16: The method of claim 1, wherein the compound specifically binds to and/or complexes with an intracellular portion or fragment of an RPTP cell adhesion molecule that is expressed by a cancer cell or another cell in the cancer cell microenvironment.

17. (canceled)

18: The method of claim 1, wherein the compound is formulated in a pharmaceutical composition for in vivo administration to the subject.

19: The method of claim 1, wherein the compound further includes a detectable moiety linked to and/or complexed with the compound, the detectable moiety including at least one of a contrast agent, imaging agent, radiolabel, semiconductor particle, or nanoparticle.

20: The method of claim 1, wherein the compound inhibits glioma cell migration in a scratch wound healing assay at least about 5% compared to glioma cells administered DMSO.

21: The method of claim 1, wherein the compound inhibits aggregation of glioma sphere formation at least about 5% compared to glioma cells administered DMSO.

22: The method of claim 1, wherein the compound inhibits aggregation of PTPμ expressing SFF9 cells at least about 5% compared to PTPμ expressing SFF9 cells administered DMSO.

23: The method of claim 1, wherein the compound inhibits PTPμ's enzymatic activity in an in vitro phosphatase assay at least about 5% compared to the in vitro phosphatase assay administered DMSO.

24: The method of claim 1, wherein the compound inhibits tumor growth at least about 5%.
